SEQUENCE LISTING (52 sequences)

SEQ ID NO: 1 - DNA, 80 bp, Artificial Sequence
  Synthetic Sequence - 40Npool
  misc_feature (21)..(60): n is a, c, g, or t
tgatagcttcagtagacgttnnnnnnnnnnnnnnnnnnnnnnnnnnnnnnnnnnnnnnnngtactctagatgcggatccc

SEQ ID NO: 2 - DNA, 37 bp, Artificial Sequence
  Synthetic Sequence - 40N-Fwd-primer
taatacgactcactatagggatccgcatctagagtac

SEQ ID NO: 3 - DNA, 20 bp, Artificial Sequence
  Synthetic Sequence - 40N-Rev-primer
tgatagcttcagtagacgtt

SEQ ID NO: 4 - RNA, 80 nt, Artificial Sequence
  Synthetic Sequence - aptamer against TGF-beta1
gggauccgcaucuagaguacuaaggguggggagacuugggccgggcagucagacgcgugaaacgucuacugaagcuauca

SEQ ID NO: 5 - RNA, 80 nt, Artificial Sequence
  Synthetic Sequence - aptamer against TGF-beta1
gggauccgcaucuagaguacaucguggcgggaaagccgccccauucucucggguccuagaaacgucuacugaagcuauca

SEQ ID NO: 6 - RNA, 80 nt, Artificial Sequence
  Synthetic Sequence - aptamer against TGF-beta1
gggauccgcaucuagaguacuuguauaaguggagggcgagacuugggaggggcgaauugaaacgucuacugaagcuauca

SEQ ID NO: 7 - RNA, 80 nt, Artificial Sequence
  Synthetic Sequence - aptamer against TGF-beta1
gggauccgcaucuagaguacgaauaguaagggaaugacucucggaccaauguauugcuauaacgucuacugaagcuauca

SEQ ID NO: 8 - RNA, 80 nt, Artificial Sequence
  Synthetic Sequence - aptamer against TGF-beta1
gggauccgcaucuagaguacgaugugcuugugcugaaauuagauuucgccgacuuucccuaacgucuacugaagcuauca

SEQ ID NO: 9 - RNA, 80 nt, Artificial Sequence
  Synthetic Sequence - aptamer against TGF-beta1
gggauccgcaucuagaguaccauaaggguggggagacuugggagagggcaaagaagacuaaacgucuacugaagcuauca

SEQ ID NO: 10 - RNA, 80 nt, Artificial Sequence
  Synthetic Sequence - aptamer against TGF-beta1
gggauccgcaucuagaguacgaugcauguuuuuauaaaguauuguuauguaaugcaucaaaacgucuacugaagcuauca

SEQ ID NO: 11 - RNA, 80 nt, Artificial Sequence
  Synthetic Sequence - aptamer against TGF-beta1
gggauccgcaucuagaguaccgcgugagcggcgucuugcuaugacguaaagaaucguuacaacgucuacugaagcuauca

SEQ ID NO: 12 - RNA, 80 nt, Artificial Sequence
  Synthetic Sequence - aptamer against TGF-beta1
gggauccgcaucuagaguaccuagaggugacuugggacgcgaguuauaagggaauaguccaacgucuacugaagcuauca

SEQ ID NO: 13 - RNA, 80 nt, Artificial Sequence
  Synthetic Sequence - aptamer against TGF-beta1
gggauccgcaucuagaguacacuagucacauugcguguacauuacucugcgcaaucgauaaacgucuacugaagcuauca

SEQ ID NO: 14 - DNA, 66 bp, Artificial Sequence
  Synthetic Sequence - 35Npool
  misc_feature (16)..(50): n is a, c, g, or t
gactgacgtcgcactnnnnnnnnnnnnnnnnnnnnnnnnnnnnnnnnnnnagctccaagttctccc

SEQ ID NO: 15 - DNA, 33 bp, Artificial Sequence
  Synthetic Sequence - 35N-Fwd-primer
taatacgactcactatagggagaacttggagct

SEQ ID NO: 16 - DNA, 15 bp, Artificial Sequence
  Synthetic Sequence - 35N-Rev-primer
gactgacgtcgcact

SEQ ID NO: 17 - RNA, 65 nt, Artificial Sequence
  Synthetic Sequence - aptamer against TGF-beta1
gggagaacuuggagcugaugucuggaguccccauauaucacguacaguguagugcgacgucaguc

SEQ ID NO: 18 - RNA, 66 nt, Artificial Sequence
  Synthetic Sequence - aptamer against TGF-beta1
gggagaacuuggagcucccccucgcacuuaauggguucuguggcuggagaaagugcgacgucaguc

SEQ ID NO: 19 - RNA, 66 nt, Artificial Sequence
  Synthetic Sequence - aptamer against TGF-beta1
gggagaacuuggagcucccccucgcauucggauuaauuugugacugcauugagugcgacgucaguc

SEQ ID NO: 20 - RNA, 66 nt, Artificial Sequence
  Synthetic Sequence - aptamer against TGF-beta1
gggagaacuuggagcugguccggaaacuggauucucucuaaaagggguaccagugcgacgucaguc

SEQ ID NO: 21 - RNA, 66 nt, Artificial Sequence
  Synthetic Sequence - aptamer against TGF-beta1
gggagaacuuggagcuccugaauaagggcggggaaacuuguggugggcuaaagugcgacgucaguc

SEQ ID NO: 22 - RNA, 66 nt, Artificial Sequence
  Synthetic Sequence - aptamer against TGF-beta1
gggagaacuuggagcuugacggcgcuacauuaugcuccaacgguacuuuauagugcgacgucaguc

SEQ ID NO: 23 - DNA, 90 bp, Artificial Sequence
  Synthetic Sequence - 50Npool
  misc_feature (21)..(70): n is a, c, g, or t
ctgactcgacgtgcaagcttnnnnnnnnnnnnnnnnnnnnnnnnnnnnnnnnnnnnnnnnnnnnnnnnnnttgaacactagtgcattccc

SEQ ID NO: 24 - DNA, 37 bp, Artificial Sequence
  Synthetic Sequence - 50N-Fwd-primer
taatacgactcactatagggaatgcactagtgttcaa

SEQ ID NO: 25 - DNA, 20 bp, Artificial Sequence
  Synthetic Sequence - 50N-Rev-primer
ctgactcgacgtgcaagctt

SEQ ID NO: 26 - RNA, 90 nt, Artificial Sequence
  Synthetic Sequence - aptamer against TGF-beta1
gggaaugcacuaguguucaaccccgaccaauagcagcccgucuuuaacuauuggaaucgcauacgggcccaagcuugcacgucgagucag

SEQ ID NO: 27 - RNA, 90 nt, Artificial Sequence
  Synthetic Sequence - aptamer against TGF-beta1
gggaaugcacuaguguucaacauuuagcaacacaagucgucccccacggcaagcaguccucaauccugacaagcuugcacgucgagucag

SEQ ID NO: 28 - RNA, 90 nt, Artificial Sequence
  Synthetic Sequence - aptamer against TGF-beta1
gggaaugcacuaguguucaauaaacacuaagugauccuccugcaagcuaugaagaacuuaacggcucguaaagcuugcacgucgagucag

SEQ ID NO: 29 - RNA, 51 nt, Artificial Sequence
  Synthetic Sequence - aptamer against TGF-beta1
gggagaacuuggagcuccugaauaagggcggggaaacuuguggugggcuaa

SEQ ID NO: 30 - RNA, 26 nt, Artificial Sequence
  Synthetic Sequence - aptamer against TGF-beta1
gggcggggaaacuuguggugggcuaa

SEQ ID NO: 31 - RNA, 33 nt, Artificial Sequence
  Synthetic Sequence - aptamer against TGF-beta1
ggcauaagggcggggaaacuuguggugggcuaa

SEQ ID NO: 32 - DNA, 66 bp, Artificial Sequence
  Synthetic Sequence - A13-Dope library
  misc_feature (22)..(25): n is a, c, g, or t
  misc_feature (29)..(30): n is a, c, g, or t
  misc_feature (34)..(39): n is a, c, g, or t
  misc_feature (44)..(44): n is a, c, g, or t
  misc_feature (48)..(51): n is a, c, g, or t
gggagaacttggagctcctgannnngggnngggnnnnnngtggngggnnnnagtgcgacgtcagtc

SEQ ID NO: 33 - DNA, 38 bp, Artificial Sequence
  Synthetic Sequence - A13-Dope-Fwd primer
taatacgactcactatagggagaacttggagctcctga

SEQ ID NO: 34 - RNA, 26 nt, Artificial Sequence
  Synthetic Sequence - consensus sequence 2
auaaggghggggagacuuguggwggg

SEQ ID NO: 35 - RNA, 66 nt, Artificial Sequence
  Synthetic Sequence - aptamer against TGF-beta1
gggagaacuuggagcuccugaauaagggaggggagacuuguggagggcaagagugcgacgucaguc

SEQ ID NO: 36 - RNA, 66 nt, Artificial Sequence
  Synthetic Sequence - aptamer against TGF-beta1
gggagaacuuggagcuccugaauaagggaggggagacuuguggagggcaaaagugcgacgucaguc

SEQ ID NO: 37 - RNA, 66 nt, Artificial Sequence
  Synthetic Sequence - aptamer against TGF-beta1
gggagaacuuggagcuccugaauaagggaggggagacuuguggugggcaagagugcgacgucaguc

SEQ ID NO: 38 - RNA, 66 nt, Artificial Sequence
  Synthetic Sequence - aptamer against TGF-beta1
gggagaacuuggagcuccugaauaaggguggggagacuuguggagggcaagagugcgacgucaguc

SEQ ID NO: 39 - RNA, 65 nt, Artificial Sequence
  Synthetic Sequence - aptamer against TGF-beta1
gggagaacuuggagcuccugaauaagggagggagacuuguggagggcaagagugcgacgucaguc

SEQ ID NO: 40 - RNA, 66 nt, Artificial Sequence
  Synthetic Sequence - aptamer against TGF-beta1
gggagaacuuggagcuccugaauaagggaggggagacuuguggagggcagaagugcgacgucaguc

SEQ ID NO: 41 - RNA, 66 nt, Artificial Sequence; identical to SEQ ID NO: 40
  Synthetic Sequence - aptamer against TGF-beta1

SEQ ID NO: 42 - RNA, 33 nt, Artificial Sequence
  Synthetic Sequence - aptamer against TGF-beta1
ggcauaagggaggggagacuuguggagggcaag

SEQ ID NO: 43 - RNA, 33 nt, Artificial Sequence
  Synthetic Sequence - aptamer against TGF-beta1
ggcauaaggguggggagacuuguggagggcaag

SEQ ID NO: 44 - RNA, 33 nt, Artificial Sequence
  Synthetic Sequence - aptamer against TGF-beta1
ggcauaagggcggggagacuuguggagggcaag

SEQ ID NO: 45 - RNA, 33 nt, Artificial Sequence
  Synthetic Sequence - aptamer against TGF-beta1
ggcauaagggaggggagacuuguggugggcaag

SEQ ID NO: 46 - RNA, 33 nt, Artificial Sequence
  Synthetic Sequence - aptamer against TGF-beta1
ggcauaaggguggggagacuuguggugggcaag

SEQ ID NO: 47 - RNA, 33 nt, Artificial Sequence
  Synthetic Sequence - aptamer against TGF-beta1
ggcauaagggcggggagacuuguggugggcaag

SEQ ID NO: 48 - RNA, 33 nt, Artificial Sequence
  Synthetic Sequence - aptamer against TGF-beta1
ggcauaagggaggggagacuuguggcggguaaa

SEQ ID NO: 49 - RNA, 25 nt, Artificial Sequence
  Synthetic Sequence - formula III-1
  misc_feature (7)..(7): n is a, c, g, or u
  misc_feature (22)..(22): n is a, c, g, or u
uaaggrnggsgaracuugkgvnrgg

SEQ ID NO: 50 - RNA, 27 nt, Artificial Sequence
  Synthetic Sequence - formula III-2
  misc_feature (9)..(9): n is a, c, g, or u
  misc_feature (24)..(24): n is a, c, g, or u
uaaguggrnggsgaracuugkgvnrgg

SEQ ID NO: 51 - RNA, 25 nt, Artificial Sequence
  Synthetic Sequence - formula III′-1
uaaggrbggsgaracuugkgvbrgg

SEQ ID NO: 52 - RNA, 27 nt, Artificial Sequence
  Synthetic Sequence - formula III′-2
uaaguggrbggsgaracuugkgvbrgg

(19) United States
(12) Patent Application Publication
(10) Pub. No.: US 2022/0282255 A1
(43) Pub. Date: Sep. 8, 2022

(54) APTAMER FOR TGF-BETA1 AND USE OF SAME

(71) Applicant: RIBOMIC INC., Tokyo (JP)

(72) Inventors: Yoshikazu NAKAMURA, Tokyo (JP); Masaki TAKAHASHI, Tokyo (JP); Yoshifumi HASHIMOTO, Tokyo (JP)

(73) Assignee: RIBOMIC INC., Tokyo (JP)

(21) Appl. No.: 17/625,308

(22) PCT Filed: Jul. 8, 2020
(86) PCT No.: PCT/JP2020/026755

§ 371 Date: Jan. 6, 2022

(30) Foreign Application Priority Data
Jul. 8, 2019 (JP) 2019-126940

(51) Int. Cl. C12N 15/115

(52) U.S. Cl. CPC C12N 15/115; C12N 2310/16

(57) ABSTRACT

The present invention aims to provide an aptamer for TGF-β1. An aptamer having four sets of consecutive G bases, and a combination of the nucleotide sequences represented by the following formula (I) and the formula (II) which binds to TGF-β1 may be useful as a medicament for preventing and/or treating various diseases involving activation of TGF-β1, or a diagnostic agent, or a labeling agent:

UAAX formula (I):

ARACUU formula (II):

wherein X is a bond or GU; and R is A or G.

### TECHNICAL FIELD

The present invention relates to an aptamer for transforming growth factor β1, a method of utilizing the same, and the like.

### BACKGROUND ART

Transforming growth factor-β (TGF-β) was initially identified as a growth factor that promotes transformation of fibroblasts (non-patent documents 1, 2). Studies in recent years have revealed that it contributes to the suppression of proliferation, cell differentiation, cell adhesion. migration, induction of apoptosis, and the like in many cell types. Therefore, TGF-β is considered to play an important role in a wide range of areas such as ontogenesis, tissue reconstruction, wound therapy, inflammation and immunity, wet metastasis of cancer, and the like. It is known that TGF-β has five isoforms having 70-80% homology in the amino acid sequence, and the first one that was discovered among these was TGF-β1. Each isoform, including TGF-β1, is secreted as a high molecular weight inactive type (latent form), activated in the vicinity of the target cell, and exerts its action. The activity of promoting production and deposition of extracellular matrix protein occupies a large part of the biological activity of TGF-β1. In various diseases that cause fibrosis (pulmonary fibrosis, liver fibrosis, scirrhous gastric cancer, etc.), the TGF-β1 level in plasma increases. In addition, the relationship with renal glomerular lesions, bone diseases, ischemic diseases, and the like is also attracting attention.

Several molecules that bind to TGF-β1 and inhibit its function have been reported heretofore. The anti-TGF-β monoclonal antibodies LY238770 (that recognizes TGF-β1) and Fresolimumab (GC1008; that recognizes TGF-31, 2, and 3) bind to the target TGF-β1 (non-patent document 3), and are expected as new therapeutic drugs for several kinds of malignant neoplasms and idiopathic pulmonary fibrosis due to the function inhibitory effects thereof (NCT00356460, NCT00923169, NCT01472731, NCT01112293, NCT01401062; non-patent document 4). In addition, a peptide that binds to TGF-β1 and exhibits a function inhibitory effect has also been reported (non-patent document 5). As small molecules, Galunisertib (LY2157299) which show a function inhibitory effect by binding to a TGF-β1 type receptor to which TGF-β1 binds, and the like have been reported.

It has been shown that antibodies specific to human TGF-β1 are effective for the treatment of TGF-β1 glomerulonephritis (non-patent document 6), neuroscars (neural scarring) (non-patent document 7), skin scar (non-patent document 8), and lung fibrosis (non-patent document 9) in animal models. Furthermore, it has been shown that antibodies against TGF-β1, 2 and 3 are effective for the models of lung fibrosis, radiation induced fibrosis (patent document 5), myelofibrosis, burn, Dupuytren's contracture, gastric ulcer and rheumatoid arthritis (non-patent document 10).

An aptamer means a nucleic acid that specifically binds to a target molecule (protein, sugar chain, hormone, etc.). It binds to a target molecule by the three-dimensional steric structure taken by single-stranded RNA (or DNA). A screening method called the SELEX method (Systematic Evolution of Ligands by Exponential Enrichment) is used for the acquisition thereof (patent documents 1-3). The aptamer obtained by the SELEX method has a chain length of about 80 nucleotides, and then the chain length is shortened by using the physiological inhibitory activity of the target molecule as an index. Furthermore, chemical modification is added to the aptamer for the purpose of improving stability in the living body, thus optimizing same as a pharmaceutical product. Aptamers have high binding properties to target molecules, and their affinity is often higher than that of antibodies having similar functions. Furthermore, aptamers are unlikely to undergo immune elimination, and adverse reactions characteristic of antibodies, such as antibody-dependent cell-mediated cytotoxicity (ADCC) and complement-dependent cytotoxicity (CDC), are reportedly unlikely to occur with the use of aptamers. From the viewpoint of drug delivery, aptamers are likely to migrate to tissues because of their molecular size of about one-tenth that of antibodies, enabling easier drug delivery to target sites. In addition, some of the small molecules of the same molecular-targeted drugs are poorly soluble, and optimization may be required for the formulation thereof. However, since aptamers are highly water-soluble, they are advantageous also on this point. Furthermore, since aptamers are produced by chemical synthesis, the cost can be reduced by mass-production. Other advantages of aptamers include long-term storage stability, heat resistance and solvent resistance. Meanwhile, the blood half-lives of aptamers are generally shorter than those of antibodies; however, this property is sometimes advantageous in view of toxicity.

As an aptamer for TGF-β, there is an aptamer developed by Gilead Sciences. Patent document 4 describes an aptamer that binds to TGF-β, which is obtained by the above-mentioned SELEX method. However, the sequences of the aptamers are different from those of the aptamers specifically shown in the present specification. In addition, this document does not suggest the aptamers specifically shown in the present specification.

### DOCUMENT LIST

### Patent Documents

patent document 1: WO 91/19813
patent document 2: WO 94/08050
patent document 3: WO 95/07364
patent document 4: WO 2005/113811
patent document 5: U.S. Pat. No. 5,616,561

### Non-Patent Documents

non-patent document 1: Roberts A B et al., Proc Natl Acad Sci USA. 1981 September; 78(9):5339-43.
non-patent document 2: Anzano M A. et al., Cancer Res. 1982 November; 42(11):4776-8.
non-patent document 3: Grutter C. et al., Proc Natl Acad Sci USA. 2008 Dec. 23; 105(51):20251-6.
non-patent document 4: Neuzillet C. et al., Pharmacol Ther. 2015 March; 147:22-31.
non-patent document 5: Gallo-Oller G. et al., Cancer Lett. 2016 Oct. 10; 381(1):67-75.
non-patent document 6: Border W A. et al., Nature. 1990 Jul. 26; 346(6282):371-4.
non-patent document 7: Logan A. et al., Eur J Neurosci. 1994 Mar. 1; 6(3):355-63.
non-patent document 8: Shah M. et al., Lancet. 1992 Jan. 25; 339(8787):213-4.
non-patent document 9: Giri S N et al., Thorax. 1993 October; 48(10):959-66.
non-patent document 10: 1554341459068_0 et al., J Exp Med. 1993 Jan. 1; 177(1):225-30.

### SUMMARY OF INVENTION

### Technical Problem

The present invention aims to provide an aptamer for TGF-β1.

### Solution to Problem

The present inventors investigated diligently to solve the problem described above and succeeded in producing aptamers that specifically bind to TGF-β1, and shown that the aptamers inhibit TGF-β1 activity. In particular, most of the aptamers were new in that they had characteristic motif sequences and four sets of consecutive G bases, and had structures completely different from those of the conventionally-known TGF aptamers.

Accordingly, the present invention provides the following:

[1] An aptamer that binds to TGF-β1, comprising four sets of consecutive G bases, and a combination of nucleotide sequences represented by the following formula (I) and the formula (II):

formula (I):
UAAX
formula (II):
ARACUU

wherein X is a bond or GU; and R is A or G.
[2] The aptamer of [1], the nucleotide sequence represented by the formula (I): UAAX is located on the most N terminal side of the four sets of G bases, and the nucleotide sequence represented by the formula (II): ARACUU is located between the second set of G bases and the third set of G bases.
[3] The aptamer of [1] or [2], comprising a nucleotide sequence represented by the following formula (III):

formula (III):
UAAXGGRNGGSGARACUUGKGVNRGG

wherein X is a bond or GU; N is any base; R is A or G; S is C or G; K is G or U; V is A, C, or G; and B is C, G, or U (only in combination that forms four sets of G bases).
[4] The aptamer of [1] or [2], comprising a nucleotide sequence represented by the following formula (III′):

formula (III′):
UAAXGGREGGSGARACUUGKGVBRGG

wherein X is a bond or GU; R is A or G; S is C or D; K is G or U; V is A, C, or G; and B is C, G, or U (only in combination that forms four sets of G bases).
[5] The aptamer of [1] or [2], comprising a nucleotide sequence represented by the following formula (III″):

formula (III″):
AUAAGGGHGGGGAGACUUGUGGWGGG

wherein W is A or U; and H is A, C, or U.
[6] The aptamer of any of [1] to [5], wherein at least one nucleotide contained in the aptamer is modified.
[7] An aptamer that binds to TGF-β1, comprising the nucleotide sequence of any of the following (a)-(c):
(a) the sequence shown in SEQ ID NO: 4-6, 9, 11, 13, 17-22, 26-29 or 31;
(b) the sequence of the above-mentioned (a), wherein one to several nucleotides are substituted, deleted, inserted, or added; or,
(c) the sequence of the above-mentioned (a) or (b), wherein at least one nucleotide is modified.
[8] The aptamer of any of [1] to [6], having a nucleotide length of not more than 55.
[9] The aptamer of any of [1] to [8], that inhibits binding between TGF-β1 and a TGF-β1 receptor.
[10] A complex comprising the aptamer of any of [1] to [9] and a functional substance.
[11] A medicament comprising the aptamer of any of [1] to [9] or the complex of [10].
[12] A method for detecting TGF-β1, comprising using the aptamer of any of [1] to [9] or the complex of [10].

### Advantageous Effects of Invention

According to the present invention, the activity of TGF-β1 can be selectively inhibited. Therefore, diseases and the like caused by overexpression of TGF-β1 can be treated according to the present invention.

### BRIEF DESCRIPTION OF DRAWINGS

FIG. 1 is a schematic drawing showing an outline of the positional relationship between a G quartet structure formed by having four sets of consecutive G bases and a specific base sequence (motif sequence), which is predicted to be a structure that the aptamer of the present invention can take. Black arrows indicate bonds or one or more bases. White arrows indicate the four sets of guanosine (sometimes to be referred to as “G sets” in the present specification) that constitute the G quartet. In this schematic drawing, a parallel type G quartet structure is depicted as an example. However, it does not deny the possibility of taking other G quartet structure types (antiparallel type, mixed type), triplexes other than quadruplexes, or other steric structures.

### DESCRIPTION OF EMBODIMENTS

The present invention is explained in detail in the following. In the present specification, nucleic acid bases are abbreviated as follows.

| symbol | meaning | explanation |
|---|---|---|
| A | A | adenine |
| C | C | cytosine |
| G | G | guanine |
| T | T | thymine |
| U | U | uracil |
| M | A or C | amino |
| R | A or G | purine |
| W | A or U | — |
| S | C or G | — |
| Y | C or U | pyrimidine |
| K | G or U | keto |
| V | A or C or G | — |
| H | A or C or U | — |
| B | C or G or U | — |
| N | A or C or G or U | — |

(The same applies when lowercase letters are used.)

The present invention provides an aptamer possessing a binding activity for TGF-β1. The aptamers of the present invention are capable of inhibiting activities of TGF-β1.

An aptamer refers to a nucleic acid molecule having a binding affinity for a particular target molecule. The aptamer can also inhibit the activity of a particular target molecule by binding to the particular target molecule. The aptamer of the present invention possesses binding activity for TGF-β1, and is capable of inhibiting a TGF-β1 activity. The aptamer of the present invention may be an RNA, a DNA, a modified nucleic acid or a mixture thereof. The aptamer of the present invention can also be in a linear or circular form.

TGF-β1 (transforming growth factor-R1) is a multifunctional cytokine, and is a protein produced by almost all cells. TGF-β1 protein is produced as a precursor polypeptide (UniProtKB-P01137, 390 amino acid residues: signal peptide (positions 1-29), LAP (positions 30-278), or mature (or active) TGF-β1 (positions 279-390)). The precursor polypeptide is cleaved by a furin-like protease to generate N-terminal LAP (latency associated protein, 249 amino acid residues) and C-terminal mature TGF-β1 (112 amino acid residues). LAP and mature TGF-β1 moieties are respectively homodimerized via disulfide bonds. Such homodimerized mature TGF-β1 and LAP bind non-covalently to form a complex. In mammals including human, TGF-β has three isoforms of β1, β2, and β3. The homology of these isoforms is 70-80%. TGF-β1 is known to have many functions such as cell proliferation, regulation of cell differentiation, induction of epithelial-mesenchymal transition, regulation of immune system through regulation of T cell differentiation, regulation of angiogenesis, promotion of extracellular matrix production, and the like. As described above, it has been reported that administration of a TGF-β1 inhibitor can treat diseases such as cancer, fibrosis, and the like.

The aptamer of the present invention shows binding activity to TGF-β1 derived from any mammal. In addition, the aptamer of the present invention can exhibit inhibitory activity against TGF-β1 derived from any mammal. Such mammals include primates (e.g., humans, monkeys), rodents (e.g., mice, rats, guinea pigs, hamsters), and companion animals, domesticated animals and work animals (e.g., dogs, cats, horses, bovines, goat, sheep, pigs), with preference given to humans. The amino acid sequence of TGF-β1 is not limited to a wild-type sequence, and may be one having one to several mutated residues in a wild-type sequence, a domain moiety thereof, or a peptide moiety thereof.

The aptamer of the present invention binds to TGF-β1 in physiological buffer solutions. Although there is no limitation on the choice of buffer solution, preference is given to buffer solutions having a pH of about 5.0-10.0. Such buffer solutions include, for example, the solution A described below (see Example 1). The aptamer of the present invention specifically binds to TGF-β1 at strength detectable by any one of the tests described below.

Binding strength may be measured using Biacore T200 (manufactured by GE Healthcare), or the like. In a method of measurement, the aptamer is first immobilized onto a sensor chip, the amount immobilized being about 1000 RU (e.g., 1500 RU, etc.). 20 μL of a TGF-β1 solution for analyte, prepared at 1 nM-200 nM (e.g., 4 nM or 10 nM, etc.), is injected, and the binding of TGF-β1 to the aptamer is detected. An RNA comprising a random nucleotide sequence of 30-100 nucleotides (e.g., 66, 80, or 90 nucleotides, etc.) is used as a negative control. If the TGF-β1 binds to the aptamer equivalently or significantly more potently compared with the control RNA, the aptamer is judged to have the capability of binding to TGF-β1.

In another method, TGF-β1 is first immobilized onto a sensor chip, the amount immobilized being about 1000 RU. 20 μL of an aptamer solution for analyte, prepared at 10 nM-200 nM (e.g., 20 nM or 100 nM, etc.), is injected, and the binding of the aptamer to TGF-β1 is detected. An RNA containing a random nucleotide sequence of 30-100 nucleotides (e.g., 66, 80, or 90 nucleotides, etc.) is used as a negative control. If the TGF-β1 binds to the aptamer equivalently or significantly more potently compared with the control RNA, the aptamer is judged to have the capability of binding to TGF-β1.

An inhibitory activity against TGF-β1 means an inhibitory potential against any activities possessed by TGF-β1. Examples of the activity of TGF-β1 include, but are not limited to, TGF-β-mediated signal transduction, extracellular matrix (ECM) deposition, inhibition of epithelial and endothelial cell proliferation, promotion of smooth muscle proliferation, induction of collagen expression, induction of expression of TGF-β, fibronectin, VEGF, and IL-11, suppression of tumor-induced immunity, promotion of angiogenesis, activation of myofibroblast, promotion of metastasis, inhibition of NK cell activity, and the like. The aptamer of the invention inhibits at least one of these activities of TGF-β1.

Whether or not an aptamer inhibits the activity of TGF-β1 can be evaluated, for example, by a cell assay system that monitors Smad signaling pathway known to be activated by the stimulation of TGF-β, as described in Examples. Briefly, it can be evaluated by the following means: Photinus luciferase equipped with SBE (Smad-binding element) in the promoter region is used as a reporter. Together with this SBE-induced photinus luciferase reporter plasmid, a renillaluciferase expression plasmid is mixed at an appropriate ratio (e.g., 20:1) as a standardized control for transfection efficiency, and transfected into HEK293 cells. The transfected HEK293 cells are re-seeded in a 96-well plate and cultured until confluent. A mixture of aptamer synthesized using TGF-β1 and T7 RNA polymerase or chemically synthesized aptamer is added thereto to an appropriate final concentration (e.g., 10 pM-100 nM, etc.), and the cells are cultured for 1-8 hr (e.g., 3 hr, etc.). Thereafter, the expression levels of photinus luciferase and renillaluciferase are confirmed by using appropriate means. By appropriately adjusting and then comparing the expression levels, whether or not the aptamer inhibits the activity of TGF-β1 can be evaluated.

In one embodiment, the aptamer of the present invention is an aptamer that binds to TGF-β1, comprising four sets of consecutive G bases, and a combination of nucleotide sequences represented by the following formula (I) and the formula (II):

formula (I):
UAAX
formula (II):
ARACUU

wherein X is a bond or GU; and R is A or G.

Note that “bond” means that a nucleotide does not exist at the position of X, and a nucleotide adjacent to the 5′ side of X (that is, adenosine 5′-phosphate) is connected to a ribonucleotide adjacent to the 3′ side of X by a phosphodiester bond.

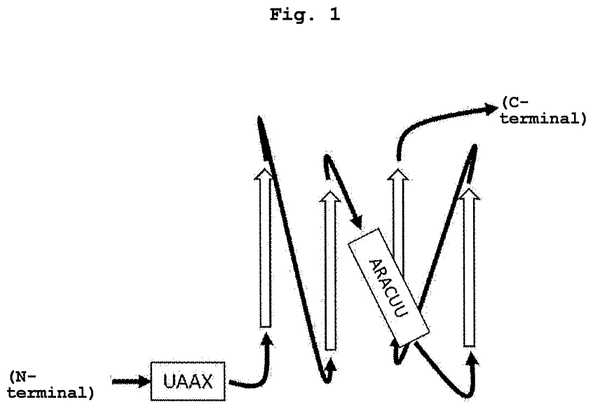

In one embodiment, the aptamer of the present invention is characterized in that it has four sets of G bases, and also has nucleotide sequences represented by the formula (I): UAAX and the formula (II): ARACUU, as mentioned above. The arrangement of the four “sets of G bases”, “the nucleotide represented by the formula (I)”, and “the nucleotide represented by the formula (II)” is not particularly limited. Preferably, the motif shown by the formula (I) UAAX and constituting the aptamer of the present invention is present at the N terminal side of the G set located on the most N terminal side (hereinafter the four G sets contained in the aptamer of the present invention are sometimes referred to as “the first G set”, “the second G set”, “the third G set”, and “the fourth G set” from the N terminal side), and the motif shown by the formula (II) ARACUU is present between the second G set and the third G set. In this schematic drawing (FIG. 1), a parallel type G quartet structure is depicted as an example. However, it does not deny the possibility of taking other G quartet structure types (antiparallel type, mixed type), triplexes other than quadruplexes, or other steric structures.

The number of G bases in the above-mentioned “set of G bases” is not particularly limited as long as it is 2 or more and the G bases are continuous. It is preferably not more than 5, and more preferably 2-4. It is not necessary for all four “sets of G bases” to have the same number of G bases, and an appropriate number of G bases can be selected as long as the aptamer of the present invention has a desired activity. However, it is desirable that the number of G bases in the “second G set” is 4, and the number of G bases in the “fourth G set” is 3.

The above-mentioned “set of G bases” and each of “the nucleotide represented by the formula (I)” and “the nucleotide represented by the formula (II)” may or may not be directly adjacent to each other as long as the aptamer of the present invention has a desired activity. The interval when they are not adjacent to each other is not particularly limited, but is desirably 1 to several bases, for example, about 1 to 9 bases, about 1 to 5 bases, and about 1 to 3 bases. There is at least one non-G base between the two “sets of G bases”.

The G quartet structure predicted as a structure that can be taken by the aptamer of the present invention is a structure well known in the art, and is an intermolecular and intramolecular quadruplex structure in DNA or RNA rich in guanosine nucleotide (G). The basic structure of the G-quadruplex structure is a plane in which four guanosine bases are tetramerized cyclically with two adjacent guanosine bases by Hoogsteen base pairs. Finally, two or three planes overlap to form a stable quadruplex structure (G-quadruplex).

The G quartet structure that the aptamer of the present invention may have is illustrated in FIG. 1. The white arrow in FIG. 1 means two or more consecutive Gs (“G set”) involved in the G quartet structure, and the black arrow means a bond or one or more bases.

An aptamer having a G quartet structure can be confirmed by a measurement means known per se.

For example, an aptamer having a G quartet structure can be confirmed by confirming a predetermined waveform by using a CD spectrum. More specifically, the aptamer is dissolved in TBS buffer (10 mM Tris-HCl, 150 mM NaCl, 5 mM KCl, pH 7.4) to prepare a sample solution, and the spectrum is measured under the condition of temperature 20° C., wavelength 200 nm-320 nm, scanning speed 100 nm/min, the number of integrations 10. An aptamer having a G quartet structure can be confirmed by the appearance of the minimum value near 240 nm and the maximum value near 260 nm in the CD spectrum.

In another embodiment, the aptamer of the present invention is an aptamer that binds to TGF-β1, comprising four sets of consecutive G bases, and a combination of the nucleotide sequence represented by the following formula (III):

formula (III):
UAAXGGRNGGSGARACUUGKGVNRGG

wherein X is a bond or GU; N is any base; R is A or G; S is C or G; K is G or U; V is A, C, or G; and B is C, G, or U (only in combination that forms four sets of G bases). The “only in combination that forms four sets of G bases” means exclusion of combinations of bases that result in a combination in which the set of G bases in the aforementioned formula is not four. Examples of the combination of bases that result in a combination in which the set of G bases in the aforementioned formula is not four include, but are not limited to, the following:

### (1) K is G,

(2) the first R is G, and the first N is G,
(3) K, V, the second N, and the third R are each G.

In one embodiment, the formula (III) may be the following sequence:

formula (III-1):
UAAGGRNGGSGARACUUGKGVNRGG (when X is a bond (SEQ
ID NO: 49)),
formula (III-2):
UAAGUGGRNGGSGARACUUGKGVNRGG (when X is GU (SEQ ID
NO: 50))

wherein N, R, S, K, V and B are as mentioned above, and are only in combination that forms four sets of G bases.

In another embodiment, the aptamer of the present invention is an aptamer that binds to TGF-β1, comprising four sets of consecutive G bases, and a combination of the nucleotide sequence represented by the following formula (III′):

formula (III′):
UAAXGGRBGGSGARACUUGKGVBRGG

wherein X is a bond or GU; R is A or G; S is C or G; K is G or U; V is A, C, or G; and B is C, G, or U (only in combination that forms four sets of G bases).

In one embodiment, the formula (III′) may be the following sequence:

formula (III′-1):
UAAGGRBGGSGARACUUGKGVBRGG (when X is a bond (SEQ
ID NO: 51)),
formula (III′-2):
UAAGUGGRBGGSGARACUUGKGVBRGG (when X is GU (SEQ
ID NO: 52))

wherein R, S, K, V and B are as mentioned above, and are only in combination that forms four sets of G bases.

In another embodiment, the aptamer of the present invention is an aptamer that binds to TGF-β1, comprising four sets of consecutive G bases, and a combination of the nucleotide sequence represented by the following formula (III″):

AUAAGGGHGGGGAGACUUGUGGWGGG formula (III″):

wherein W is A or U; and H is A, C, or U) (SEQ ID NO: 34)

In another embodiment, the aptamer of the present invention may also include the following aptamers having an activity of binding to TGF-β1 and/or an activity of inhibiting the biological activity of TGF-β1:

(a) aptamer containing the base sequence shown in SEQ ID NO: 4-6, 9, 11, 13, 17-22, 26-29 or 31;
(b) aptamer containing the sequence wherein one to several (e.g., 1, 2, 3, 4, or 5) nucleotides are substituted, deleted, inserted, or added in the above-mentioned (a); or,
(c) aptamer containing the sequence, wherein at least one nucleotide is modified in the above-mentioned (a) or (b).

The aptamers recited here may include an aptamer free of the aforementioned common motif (i.e., SEQ ID NO: 5, 11, 13, 18, 19, 20, 22, 26, and 27).

In another embodiment of the present invention, the aptamer of the present invention is the following aptamer:

(a) aptamer consisting of the base sequence shown in SEQ ID NO: 4-6, 9, 11, 13, 17-22, 26-29 or 31;
(b) aptamer consisting of the sequence wherein one to several (e.g., 1, 2, 3, 4, or 5) nucleotides are substituted, deleted, inserted, or added in the above-mentioned (a); or,
(c) aptamer consisting of the sequence wherein at least one nucleotide is modified in the above-mentioned (a) or (b).

The length of the aptamer of the present invention is not limited, and can usually be about 25 to about 200 nucleotides, and can be, for example, not more than about 100 nucleotides, preferably not more than about 55 nucleotides, more preferably not more than about 45 nucleotides, most preferably not more than about 35 nucleotides. When the total number of nucleotides is smaller, chemical synthesis and mass-production will be easier, and there is a major advantage in terms of cost. It is also thought that chemical modification is easy, stability in the body is high, and toxicity is low. On the other hand, the length of the aptamer of the present invention is generally not less than about 25 nucleotides, preferably not less than about 28 nucleotides, more preferably not less than about 29 nucleotides, particularly preferably not less than about 30 nucleotides. When the total number of nucleotides is too small, the common sequence explained below cannot be contained, and the potential tertiary structure becomes unstable and the activity may be lost in some cases.

The aptamer of the present invention inhibits the activity of TGF-β1 by specifically binding to TGF-β1. The aptamer of the present invention may bind to any part of TGF-β1 and inhibit the activity of TGF-β1 by any action mechanism as long as it can inhibit the activity of TGF-β1. In one embodiment, the aptamer of the present invention can inhibit the activity of TGF-β1 by inhibiting the binding between TGF-β1 and the receptor of TGF-β1 by binding to TGF-β1. It is clear that the aptamer known to inhibit the activity of TGF-β1 is an aptamer that binds to TGF-β1, even without confirming the binding to TGF-β1.

The aptamer of the present invention may be one wherein a sugar residue (e.g., ribose) of each nucleotide has been modified to increase the TGF-β1 binding activity, stability, drug deliverability and the like. As examples of the site to be modified in a sugar residue, one having the oxygen atom at the 2′-position, 3′-position and/or 4′-position of the sugar residue replaced with another atom, and the like can be mentioned. As examples of the modification, fluorination, O-alkylation (e.g., O-methylation, O-ethylation), O-arylation, S-alkylation (e.g., S-methylation, S-ethylation), S-arylation, and amination (e.g., —NH₂) can be mentioned. Such alterations in the sugar residue can be performed by a method known per se (see, for example, Sproat et al., (1991) Nucle. Acid. Res. 19, 733-738; Cotton et al., (1991) Nucl. Acid. Res. 19, 2629-2635; Hobbs et al., (1973) Biochemistry 12, 5138-5145).

In one embodiment, each of the nucleotides contained in the aptamer of the present invention, whether identical or different, can be a nucleotide comprising a hydroxyl group at the 2′ position of ribose (e.g., ribose of pyrimidine nucleotide, ribose of purine nucleotide) (i.e., an unsubstituted nucleotide) or a nucleotide substituted by any atom or group at the 2′ position of ribose. As examples of such any atom or group, a nucleotide substituted by a hydrogen atom, a fluorine atom or an —O-alkyl group (e.g., —O-Me group), an —O-acyl group (e.g., —O—COMe group (sometimes to be also denoted as “OMe group”)), or an amino group (e.g., —NH₂ group) can be mentioned.

In the aptamer of the present invention, all pyrimidine nucleotides can be nucleotides substituted by a fluorine atom, or nucleotides substituted by any atom or group mentioned above, preferably an atom or group selected from the group consisting of a hydrogen atom, a hydroxyl group and a methoxy group whether identical or not, at the 2′ position of ribose.

In the aptamers of the present invention, all purine nucleotides can be nucleotides substituted by a hydroxyl group, or nucleotides substituted by any atom or group mentioned above, preferably an atom or a group selected from the group consisting of a hydrogen atom, a methoxy group, and a fluorine atom, whether identical or not, at the 2′-position of ribose.

The aptamer of the present invention can also be one wherein all nucleotides identically comprise a hydroxyl group, or any atom or group mentioned above, for example, the identical group selected from the group consisting of a hydrogen atom, a fluorine atom, a hydroxyl group and a methoxy group, at the 2′ position of ribose.

The modification of the aptamer of the present invention can further be performed by adding to an end a polyethyleneglycol, amino acid, peptide, inverted dT, nucleic acid, nucleosides, Myristoyl, Lithocolic-oleyl, Docosanyl, Lauroyl, Stearoyl, Palmitoyl, Oleoyl, Linoleoyl, other lipids, steroids, cholesterol, caffeine, vitamins, pigments, fluorescent substances, anticancer agent, toxin, enzymes, radioactive substance, biotin and the like. For such modifications, see, for example, U.S. Pat. Nos. 5,660,985 and 5,756,703.

Herein, in this specification, the nucleotides constituting the aptamer are assumed to be RNAs (i.e., the sugar groups are assumed to be ribose) in describing how the sugar groups are modified in the nucleotides. However, this does not mean that DNA is exempted from the aptamer-constituting nucleotides, and a modification of RNA should read as a modification of DNA as appropriate. When the nucleotide constituting the aptamer is DNA, for example, substitution of the hydroxyl group at the 2′ position of ribose by X should read as a substitution of one hydrogen atom at the 2′ position of deoxyribose by X.

The aptamer of the present invention can be synthesized by the method disclosed herein or by a method known per se in the art. A method of synthesis employs RNA polymerase. A DNA having a desired sequence and a promoter sequence of RNA polymerase is chemically synthesized, which, as a template, is transcribed by a publicly known method to obtain the desired RNA. The aptamer of the present invention can also be synthesized using DNA polymerase. A DNA having a desired sequence is chemically synthesized, which, as a template, is amplified by a method of public knowledge known as the polymerase chain reaction (PCR). This is rendered single-stranded by a publicly known method of polyacrylamide electrophoresis or enzyme treatment. When synthesizing a modified aptamer, elongation reaction efficiency can be increased by using a polymerase mutated at a particular site. The aptamer thus obtained can easily be purified by a publicly known method.

An aptamer can be synthesized in large amounts by chemical synthetic methods such as the amidite method and the phosphoramidite method. These synthetic methods are well known, as described in Nucleic Acid (Vol. 2) [1] Synthesis and Analysis of Nucleic Acid (edited by Yukio Sugiura, published by Hirokawa Publishing Company) and the like. Practically, a synthesizer such as OligoPilot100 or OligoProcess (manufactured by GE Healthcare Bioscience) is used. The aptamer thus synthesized can be purified by a method known per se such as chromatography.

Provided that an active group such as an amino group is introduced to an aptamer during the process of chemical synthesis by the phosphoramidite method or the like, a functional substance can be added after the synthesis. For example, by introducing an amino group to an end of the aptamer, it is possible to condense a polyethylene glycol chain incorporating a carboxyl group.

An aptamer binds to the target molecule in a wide variety of binding modes, such as ionic bonds based on the negative charge of the phosphate group, hydrophobic bonds and hydrogen bonds based on ribose, and hydrogen bonds and stacking interaction based on nucleic acid bases. In particular, ionic bonds based on the negative charge of the phosphate group, which are present in the same number as the number of constituent nucleotides, are strong, and bind to the positive charge of lysine and arginine being present on the surface of protein. For this reason, nucleic acid bases not involved in the direct binding to the target molecule can be substituted. Regarding modifications of the 2′-position of ribose, the functional group at the 2′-position of ribose infrequently interacts directly with the target molecule, but in many cases, it is of no relevance, and can be substituted by another modified molecule. Hence, an aptamer, unless the functional group involved in the direct binding to the target molecule is substituted or deleted, often retains the activity thereof. It is also important that the overall three-dimensional structure does not change widely.

An aptamer can be prepared by utilizing the SELEX method or an improved version thereof (for example, Ellington et al., (1990) Nature, 346, 818-822; Tuerk et al., (1990) Science, 249, 505-510). In the SELEX method, by rendering the selection criteria more rigorous by increasing the number of rounds or using a competing substance, an aptamer exhibiting a stronger binding potential for the target molecule is concentrated and selected. Hence, by adjusting the number of rounds of SELEX and/or changing the competitive condition, aptamers with different binding forces, aptamers with different binding modes, and aptamers with the same binding force or binding mode but different base sequences can be obtained in some cases. The SELEX method comprises a process of amplification by PCR; by causing a mutation by using manganese ions and the like in the process, it is possible to perform SELEX with higher diversity.

The aptamers obtained by SELEX are nucleic acids that exhibit high affinity for the target molecule, but this does not mean inhibiting a bioactivity of the target molecule.

Based on an active aptamer thus selected, SELEX can be performed to acquire an aptamer possessing higher activity. Specifically, after preparing a template wherein an aptamer with a determined sequence is partially randomized or a template doped with about 10 to 30% of random sequences, SELEX is performed again.

An aptamer obtained by SELEX has a length of about 80 nucleotides, and this is difficult to prepare as a pharmaceutical as it is. Hence, it is necessary to repeat try-and-error efforts to shorten the aptamer to a length of about 50 nucleotides or less enabling easy chemical synthesis. Depending on the primer design for an aptamer obtained by SELEX, the ease of the subsequent minimization operation changes. Unless the primer is designed successfully, subsequent development will be impossible even if an aptamer with activity is selected by SELEX.

Aptamers are easily modifiable because they permit chemical synthesis. For aptamers, by predicting the secondary structure using the MFOLD program, or by predicting the steric structure by X-ray analysis or NMR analysis, it is possible to predict to some extent which nucleotide can be substituted or deleted, and where to insert a new nucleotide. A predicted aptamer with the new sequence can easily be chemically synthesized, and it can be determined whether or not the aptamer retains the activity using an existing assay system.

If a region important to the binding of the aptamer obtained with the target molecule is identified by repeated try-and-error efforts as described above, the activity remains unchanged in many cases even when a new sequence is added to both ends of the sequence. The length of the new sequence is not particularly limited.

Those of ordinary skill in the art can make a wide range of design or alterations of modifications, like sequences.

As stated above, aptamers permit a wide range of design or alterations.

The present invention also provides a complex comprising the aptamer of the present invention and a functional substance bound thereto (hereinafter sometimes referred to as “the complex of the present invention”). The bond between the aptamer and the functional substance in the complex of the present invention can be a covalent bond or a non-covalent bond. The complex of the present invention can be one wherein the aptamer of the present invention and one or more (e.g., 2 or 3) of functional substances of the same kind or different kinds are bound together. The functional substance is not particularly limited, as far as it newly confers a certain function to an aptamer of the present invention, or is capable of changing (e.g., improving) a certain characteristic which an aptamer of the present invention can possess. As examples of the functional substance, proteins, peptides, amino acids, lipids, sugars, monosaccharides, polynucleotides, and nucleotides can be mentioned. As examples of the functional substance, affinity substances (e.g., biotin, streptavidin, polynucleotides possessing affinity for target complementary sequence, antibodies, glutathione Sepharose, histidine), substances for labeling (e.g., fluorescent substances, luminescent substances, radioisotopes), enzymes (e.g., horseradish peroxidase, alkaline phosphatase), drug delivery vehicles (e.g., liposome, microspheres, peptides, polyethyleneglycols), drugs (e.g., those used in missile therapy such as calicheamycin and duocarmycin; nitrogen mustard analogues such as cyclophosphamide, melphalan, ifosfamide or trofosfamide; ethylenimines such as thiotepa; nitrosoureas such as carmustine; alkylating agents such as temozolomide or dacarbazine; folate-like antimetabolites such as methotrexate or raltitrexed; purine analogues such as thioguanine, cladribine or fludarabine; pyrimidine analogues such as fluorouracil, tegafur or gemcitabine; vinca alkaloids such as vinblastine, vincristine or vinorelbine and analogues thereof; podophyllotoxin derivatives such as etoposide, taxans, docetaxel or paclitaxel; anthracyclines such as doxorubicin, epirubicin, idarubicin and mitoxantrone, and analogues thereof; other cytotoxic antibiotics such as bleomycin and mitomycin; platinum compounds such as cisplatin, carboplatin and oxaliplatin; pentostatin, miltefosine, estramustine, topotecan, irinotecan and bicalutamide), and toxins (e.g., ricin toxin, liatoxin and Vero toxin) can be mentioned. These functional molecules are finally removed in some cases. Furthermore, the molecules may be peptides that can be recognized and cleaved by enzymes such as thrombin, matrix metalloproteinase (MMP), and Factor X, and may be polynucleotides that can be cleaved by nucleases or restriction endonuclease.

The aptamer or the complex of the present invention can be used as, for example, a pharmaceutical or a diagnostic reagent, a testing reagent or an analytical reagent.

The aptamer and the complex of the present invention can selectively inhibit the activity of TGF-β1. TGF-β1 is a multifunctional cytokine involved in cell proliferation and differentiation, embryogenic development, extracellular matrix formation, bone development, wound healing, hematopoiesis, and immune response and inflammation response. Therefore, overexpression of TGF-β1 is considered to relate to a number of conditions in humans, such as fibrotic diseases, cancer, immune-mediated diseases, wound healing, renal disease, and the like. Accordingly, the aptamer and the complex of the present invention are also useful as medicaments for treating or preventing these diseases.

The aptamer and the complex of the present invention may be useful for the treatment or prophylaxis of fibrous diseases such as glomerulonephritis, neuroscars, skin scar, lung fibrosis, radiation induced fibrosis, hepatic fibrosis, myelofibrosis, and the like.

The aptamer and the complex of the present invention may be useful for the treatment or prophylaxis of cancers such as breast cancer, prostate cancer, ovarian cancer, gastric cancer, kidney cancer, pancreatic cancer, colon rectal cancer, skin cancer, lung cancer, cervix cancer, bladder cancer, glioma, mesothelioma, leukemia, sarcoma, and the like.

The aptamer and the complex of the present invention may be useful for enhancing the immune response to macrophage-mediated infections and reducing immunosuppression caused by tumor, AIDS, and the like.

In addition, the aptamer and the complex of the present invention may be useful for treating wounds such as systemic sclerosis, postoperative adhesion, keloid, hypertrophic scar, cornea damage, cataract, Peyronie's disease, cirrhosis, scar after myocardial infarction, post-angioplastic restenosis, scar after subarachnoid hemorrhage, biliary cirrhosis (including sclerosing cholangitis), and the like.

In addition, the aptamer and the complex of the present invention may be useful for the treatment or prophylaxis of renal diseases such as diabetic (type I and type II) renopathy, radiation-induced nephropathy, obstructive nephropathy, hereditary renal disease (e.g., polycystic kidney, medullary sponge kidney, horseshoe kidney), glomerulonephritis, nephrosclerosis, kidney calcification, systemic lupus erythematosus, Sjogren's syndrome, Buerger disease, systemic or glomerular hypertension, renal tubular interstitial nephritis, renal tubular acidosis, kidney tuberculosis, renal infarction, and the like.

The aptamer and the complex of the present invention are capable of binding specifically to TGF-31. Therefore, in another embodiment of the present invention, the aptamer and the complex of the present invention may be useful as probes for TGF-β1 detection. The probes are useful in in vivo imaging of TGF-β1, measurements of blood concentrations of TGF-β1, tissue staining of TGF-β1, ELISA of TGF-β1 and the like. The probes may also be useful as diagnostic reagents, testing reagents, analytical reagents and the like for diseases involving TGF-β1 (cancer, fibrosis, and the like).

Based on their specific binding to TGF-β1, the aptamer and the complex of the present invention can be used as ligands for purification of TGF-β1.

The aptamer and the complex of the present invention can be used as drug delivery vehicles.

The medicament of the present invention containing the aptamer of the present invention or a complex containing the aptamer of the present invention can be one formulated with a pharmaceutically acceptable carrier. As examples of the pharmaceutically acceptable carrier, excipients such as sucrose, starch, mannit, sorbit, lactose, glucose, cellulose, talc, calcium phosphate, and calcium carbonate; binders such as cellulose, methylcellulose, hydroxylpropylcellulose, polypropylpyrrolidone, gelatin, gum arabic, polyethylene glycol, sucrose, and starch; disintegrants such as starch, carboxymethylcellulose, hydroxylpropylstarch, sodium-glycol-starch, sodium hydrogen carbonate, calcium phosphate, and calcium citrate; lubricants such as magnesium stearate, Aerosil, talc, and sodium lauryl sulfate; flavoring agents such as citric acid, menthol, glycyrrhizin-ammonium salt, glycine, and orange powder; preservatives such as sodium benzoate, sodium hydrogen sulfite, methylparaben, and propylparaben; stabilizers such as citric acid, sodium citrate, and acetic acid; suspending agents such as methylcellulose, polyvinylpyrrolidone, and aluminum stearate; dispersing agents such as surfactants; diluents such as water, physiological saline, and orange juice; base waxes such as cacao butter, polyethylene glycol, and kerosene; and the like can be mentioned, but these are not limitative.

There is no limitation on the route of administration of the pharmaceutical of the present invention, which can be administered by, for example, oral administration and parenteral administration.

Preparations suitable for oral administration are a liquid prepared by dissolving an effective amount of ligand in a diluent such as water, physiological saline, or orange juice; capsules, sachets or tablets comprising an effective amount of ligand in solid or granular form; a suspension prepared by suspending an effective amount of active ingredient in an appropriate dispersant; an emulsion prepared by dispersing and emulsifying a solution of an effective amount of active ingredient in an appropriate dispersant; C10, which promotes the absorption of water-soluble substances, and the like.

The pharmaceutical of the present invention can be coated by a method known per se for the purpose of taste masking, enteric dissolution, sustained release and the like as required. As examples of coating agents used for the coating, hydroxypropylmethylcellulose, ethylcellulose, hydroxymethylcellulose, hydroxypropylcellulose, polyoxyethylene glycol, Tween 80, Pluronic F68, cellulose acetate phthalate, hydroxypropylmethylcellulose phthalate, hydroxymethylcellulose acetate succinate, Eudragit (manufactured by Rohm, Germany, methacrylic acid/acrylic acid copolymer), pigments (e.g., red iron oxide, titanium dioxide and the like) and the like are used. The pharmaceutical may be a rapid-release preparation or sustained-release preparation.

As preparations suitable for parenteral administration (for example, intravenous administration, subcutaneous administration, intramuscular administration, topical administration, intraperitoneal administration, intranasal administration and the like), aqueous and non-aqueous isotonic sterile injectable liquids are available, which may comprise an antioxidant, a buffer solution, a bacteriostatic agent, an isotonizing agent and the like. Aqueous and non-aqueous sterile suspensions can also be mentioned, which may comprise a suspending agent, a solubilizer, a thickener, a stabilizer, an antiseptic and the like. The preparation can be included in a container such as an ampoule or a vial in a unit dosage volume or in several divided doses. An active ingredient and a pharmaceutically acceptable carrier can also be freeze-dried and stored in a state that may be dissolved or suspended in an appropriate sterile vehicle just before use.

Sustained-release preparations are also suitable preparations. Dosage forms of sustained-release preparations include sustained release from carriers or containers embedded in the body, such as artificial bones, biodegradable bases or non-biodegradable sponges, bags and the like. Devices for continuous or intermittent, systemic or topical delivery from outside the body, such as drug pumps and osmotic pressure pumps, are also included in the scope of sustained-release preparations. Biodegradable bases include liposome, cationic liposome, poly(lactic-co-glycolic) acid (PLGA), atherocollagen, gelatin, hydroxyapatite, polysaccharide sizofiran.

In addition to liquid injections, suspensions and sustained-release preparations, inhalants suitable for transpulmonary administration, ointments suitable for percutaneous administration, and the like are acceptable.

In the case of an inhalant, an active ingredient in a freeze-dried state is micronized and administered by inhalation using an appropriate inhalation device. An inhalant can be formulated as appropriate with a conventionally used surfactant, oil, seasoning, cyclodextrin or derivative thereof and the like as required. An inhalant can be produced according to a conventional method. Specifically, an inhalant can be produced by powdering or liquefying the aptamer or complex of the present invention, blending it in an inhalation propellant and/or carrier, and filling it in an appropriate inhalation vessel. When the above-described aptamer or complex of the present invention is a powder, an ordinary mechanical powder inhalator can be used; in the case of a liquid, an inhalator such as a nebulizer can be used. Here, as the propellant, conventionally known one can be widely used; flon-series compounds such as flon-11, flon-12, flon-21, flon-22, flon-113, flon-114, flon-123, flon-142c, flon-134a, flon-227, flon-C318, and 1,1,1,2-tetrafluoroethane, hydrocarbons such as propane, isobutane, and n-butane, ethers such as diethyl ether, compressed gases such as gaseous nitrogen and gaseous carbon dioxide and the like can be mentioned.

Here, as examples of the surfactant, oleic acid, lecithin, diethyleneglycol dioleate, tetrahydroflufuryl oleate, ethyl oleate, isopropyl myristate, glyceryl trioleate, glyceryl monolaurate, glyceryl monooleate, glyceryl monostearate, glyceryl monolysinoate, cetyl alcohol, stearyl alcohol, polyethyleneglycol 400, cetylpyridinium chloride, sorbitan trioleate (trade name Span 85), sorbitan monooleate (trade name Span 80), sorbitan monolaurate (trade name Span 20), polyoxyethylene hydrogenated castor oil (trade name HCO-60), polyoxyethylene (20) sorbitan monolaurate (trade name Tween 20), polyoxyethylene (20) sorbitan monooleate (trade name Tween 80), lecithin of natural resource origin (trade name EPICLON), oleylpolyoxyethylene (2) ether (trade name Brij 92), stearyl polyoxyethylene (2) ether (trade name Brij 72), lauryl polyoxyethylene (4) ether (trade name Brij 30), oleylpolyoxyethylene (2) ether (trade name Genapol 0-020), block copolymer of oxyethylene and oxypropylene (trade name Synperonic) and the like can be mentioned. As examples of the oil, corn oil, olive oil, cottonseed oil, sunflower oil and the like can be mentioned. In the case of an ointment, an appropriate pharmaceutically acceptable base (yellow petrolatum, white petrolatum, paraffin, plastibase, silicone, white ointment, beeswax, lard, vegetable oils, hydrophilic ointment, hydrophilic petrolatum, purified lanolin, hydrolyzed lanolin, water-absorbing ointment, hydrophilic plastibase, macrogol ointment and the like) is blended with an aptamer of the present invention, which is the active ingredient, and used as a preparation.

The dosage of the pharmaceutical of the present invention varies depending on the kind and activity of active ingredient, seriousness of disease, animal species being the subject of administration, drug tolerability of the subject of administration, body weight, age and the like, and the usual dosage, based on the amount of active ingredient per day for an adult, can be about 0.0001 to about 100 mg/kg, for example, about 0.0001 to about 10 mg/kg, preferably about 0.005 to about 1 mg/kg.

The present invention also provides a solid phase carrier having the aptamer and/or the complex of the present invention immobilized thereon. As examples of the solid phase carrier, a substrate, a resin, a plate (e.g., multiwell plate), a filter, a cartridge, a column, and a porous material can be mentioned. The substrate can be one used in DNA chips, protein chips and the like; for example, nickel-PTFE (polytetrafluoroethylene) substrates, glass substrates, apatite substrates, silicon substrates, alumina substrates and the like, and substrates prepared by coating these substrates with a polymer and the like can be mentioned. As examples of the resin, agarose particles, silica particles, a copolymer of acrylamide and N,N′-methylenebisacrylamide, polystyrene-crosslinked divinylbenzene particles, particles of dextran crosslinked with epichlorohydrin, cellulose fiber, crosslinked polymers of aryldextran and N,N′-methylenebisacrylamide, monodispersed synthetic polymers, monodispersed hydrophilic polymers, Sepharose, Toyopearl and the like can be mentioned, and also resins prepared by binding various functional groups to these resins were included. The solid phase carrier of the present invention can be useful in, for example, purification of TGF-β1, and detection and quantification of TGF-β1.

The aptamer and/or the complex of the present invention can be immobilized onto a solid phase carrier by a method known per se. For example, a method that introduces an affinity substance (e.g., those described above) or a predetermined functional group into the aptamer and/or the complex of the present invention, and then immobilizing the aptamer or complex onto a solid phase carrier via the affinity substance or predetermined functional group can be mentioned. The present invention also provides such methods. The predetermined functional group can be a functional group that can be subjected to a coupling reaction; for example, an amino group, a thiol group, a hydroxyl group, and a carboxyl group can be mentioned. The present invention also provides an aptamer having such a functional group introduced thereto.

The present invention also provides a method of purifying and concentrating TGF-β1. In particular, the present invention makes it possible to separate TGF-β1 from the proteins of other family proteins. The method of purification and concentration of the present invention can comprise adsorbing TGF-β1 to the solid phase carrier of the present invention, and eluting the adsorbed TGF-β1 with an eluent. Adsorption of TGF-β1 to the solid phase carrier of the present invention can be achieved by a method known per se. For example, a TGF-β1-containing sample (e.g., bacterial or cell culture or culture supernatant, blood) is introduced into the solid phase carrier of the present invention or a composition containing the same. For elution of TGF-β1, an eluent can be appropriately selected in consideration of the known properties of TGF-β1. The method of purification and concentration of the present invention can further comprise washing the solid phase carrier using a washing solution after TGF-β1 adsorption. The washing solution can be appropriately selected in consideration of the known properties of TGF-β1. The method of purification and concentration of the present invention can still further comprise heating the solid phase carrier. This step enables the regeneration and sterilization of the solid phase carrier.

The present invention also provides a method of detecting and quantifying TGF-β1. In particular, the present invention makes it possible to detect and quantify TGF-β1 separately from the proteins of other family proteins. The method of detection and quantitation of the present invention can comprise measuring TGF-β1 by utilizing the aptamer of the present invention (e.g., by the use of the complex and solid phase carrier of the present invention). The method of detecting and quantifying TGF-β1 can be performed in the same manner as an immunological method, except that the aptamer of the present invention is used in place of an antibody. Therefore, by using the aptamer of the present invention as a probe in place of an antibody, in the same manner as such methods as enzymeimmunoassay (EIA) (e.g., direct competitive ELISA, indirect competitive ELISA, sandwich ELISA), radioimmunoassay (RIA), fluorescent immunoassay (FIA), Western blot technique, immunohistochemical staining method, and cell sorting method, detection and quantitation can be performed. The aptamer of the present invention can also be used as a molecular probe for PET and the like. These methods can be useful in, for example, measuring TGF-β1 contents in living organisms or biological samples, and in diagnosing a disease associated with TGF-β1.

The disclosures in all publications mentioned herein, including patents and patent application specifications, are incorporated by reference herein in the present invention to the extent that all of them have been given expressly.

The present invention is hereinafter described in more detail by means of the following Examples, which, however, never limit the scope of the invention.

### EXAMPLE

### [Example 1] Production of RNA Aptamers that Bind Specifically to TGF-β1—1

RNA aptamers that bind specifically to TGF-β1 were produced using the SELEX method. SELEX was performed with improvements of the method of Ellington et al. (Ellington and Szostak, Nature 346, 818-822, 1990) and the method of Tuerk et al. (Tuerk and Gold, Science 249, 505-510, 1990). TGF-β1 (Recombinant Human TGF-β1, manufactured by Peprotech, hereinafter denoted as TGF-β1) immobilized on NHS-activated Sepharose™ 4 Fast Flow (manufactured by GE Healthcare) carrier was used as a target substance. The carrier on which TGF-β1 was immobilized was obtained by activating the carrier with 1 mM hydrochloric acid, mixing the both, and reacting them for about 3 hr at room temperature. The amount immobilized was confirmed by examining the TGF-β1 solution before immobilization and the supernatant just after immobilization by SDS-PAGE. As a result of the SDS-PAGE, no band of TGF-β1 was detected in the supernatant; it was confirmed that nearly all of the TGF-β1 used had been coupled. This means that about 40 pmol of TGF-β1 was immobilized to about 1 μL of the resin.

The RNA of the random sequence used in the first round (40N) was obtained by transcribing a chemically synthesized DNA using T7 RNA polymerase (Y639F). The RNA obtained by this method has the 2′-position of ribose of the pyrimidine nucleotide fluoro-substituted. The following DNA of 80 nucleotides length, having a primer sequence at each end of a 40-nucleotide random sequence, was used as a DNA template. The DNA template and primers used were prepared by chemical synthesis.

DNA template sequence:
(SEQ ID NO: 1)
5′-TGATAGCTTCAGTAGACGTTNNNNNNNNNNNNNNNNNNNNNNNNNNN
NNNNNNNNNNNNNGTACTCTAGATGCGGATCCC-3′
primer Fwd:
(SEQ ID NO: 2)
5′-TAATACGACTCACTATAGGGATCCGCATCTAGAGTAC-3′
primer Rev:
(SEQ ID NO: 3)
5′-TGATAGCTTCAGTAGACGTT-3′

N in the DNA template (SEQ ID NO: 1) is any combination of nucleotides (A, G, C or T). The primer Fwd contains a promoter sequence of T7 RNA polymerase.

The RNA pool was added to the TGF-β1-immobilized carrier, and allowed to stand at room temperature for 30 min. Then, to remove the RNA not bound to TGF-β1, the resin was washed with solution A. Here, the solution A was a mixed solution of 145 mM sodium chloride, 5.4 mM potassium chloride, 1.8 mM calcium chloride, 0.8 mM magnesium chloride, 20 mM Tris (pH 7.6), and 0.05% Tween20. The RNA bound to TGF-β1 was heated at 90° C. for 5 min with the addition of solution B as an eluent, and recovered from the supernatant. Here, the solution B was a mixture of 7M Urea, 5 mM EDTA, and 0.1 M Tris (pH 7.6). The recovered RNA was amplified by RT-PCR and transcribed using T7 RNA polymerase (Y639F), and this was used as the pool for the next round. With this procedure taken as 1 round, the same operation was performed plural times. After completion of SELEX, the PCR product was cloned into pGEM-T Easy vector (manufactured by Promega), and the Escherichia coli strain DH5α (manufactured by TAKARA) was transformed therewith. After the plasmid was extracted from a single colony, the base sequences of clones were determined using a DNA sequencer (outsourced to FASMAC).

After 5 rounds of SELEX, the sequences of 88 clones were sequenced, and sequence convergence was found. SEQ ID NOs: 4 to 13 were obtained as sequences from which two or more clones were obtained.

The nucleotide sequences of SEQ ID NOs: 4-13 are shown in the following. Unless particularly indicated, the individual sequences listed in the Examples are shown in the direction of 5′ to 3′, where in each nucleotide, purine (A and G) is 2′-OH (natural RNA type) and pyrimidine (U and C) is a 2′-fluoromodified product. The primer binding sequence is shown in lower case.

SEQ ID NO: 4:
gggauccgcaucuagaguacUAAGGGUGGGGAGACUUGGGCCGGGCAGUC
AGACGCGUGAaacgucuacugaagcuauca
SEQ ID NO: 5:
gggauccgcaucuagaguacAUCGUGGCGGGAAAGCCGCCCCAUUCUCUC
GGGUCCUAGAaacgucuacugaagcuauca
SEQ ID NO: 6:
gggauccgcaucuagaguacUUGUAUAAGUGGAGGGCGAGACUUGGGAGG
GGCGAAUUGAaacgucuacugaagcuauca
SEQ ID NO: 7:
gggauccgcaucuagaguacGAAUAGUAAGGGAAUGACUCUCGGACCAAU
GUAUUGCUAUaacgucuacugaagcuauca
SEQ ID NO: 8:
gggauccgcaucuagaguacGAUGUGCUUGUGCUGAAAUUAGAUUUCGCC
GACUUUCCCUaacgucuacugaagcuauca
SEQ ID NO: 9:
gggauccgcaucuagaguacCAUAAGGGUGGGGAGACUUGGGAGAGGGCA
AAGAAGACUAaacgucuacugaagcuauca
SEQ ID NO: 10:
gggauccgcaucuagaguacGAUGCAUGUUUUUAUAAAGUAUUGUUAUGU
AAUGCAUCAAaacgucuacugaagcuauca
SEQ ID NO: 11:
gggauccgcaucuagaguacCGCGUGAGCGGCGUCUUGCUAUGACGUAAA
GAAUCGUUACaacgucuacugaagcuauca
SEQ ID NO: 12:
gggauccgcaucuagaguacCUAGAGGUGACUUGGGACGCGAGUUAUAAG
GGAAUAGUCCaacgucuacugaagcuauca
SEQ ID NO: 13:
gggauccgcaucuagaguacACUAGUCACAUUGCGUGUACAUUACUCUGC
GCAAUCGAUAaacgucuacugaagcuauca

### [Example 2] Production of RNA Aptamers that Bind Specifically to TGF-β1—2

The same SELEX as in Example 1 was performed using a template having a random sequence of 35 nucleotides and a primer sequence different from that used in Example 1. TGF-β1 (Recombinant Human TGF-β1, manufactured by Peprotech) immobilized on NHS-activated Sepharose™ 4 Fast Flow (manufactured by GE Healthcare) carrier was used as a target substance of SELEX. The sequences of the template and primers used are shown below. The DNA template and the primers were produced by chemical synthesis.

DNA template sequence:
(SEQ ID NO: 14)
5′-GACTGACGTCGCACTNNNNNNNNNNNNNNNNNNNNNNNNNNNNNNNN
NNNAGCTCCAAGTTCTCCC-3′
primer Fwd:
(SEQ ID NO: 15)
5′-TAATACGACTCACTATAGGGAGAACTTGGAGCT-3′
primer Rev:
(SEQ ID NO: 16)
5′-GACTGACGTCGCACT-3′

N in the DNA template (SEQ ID NO: 14) is any combination of nucleotides (A, G, C or T). The primer Fwd contains a promoter sequence of T7 RNA polymerase.

After 7 rounds of SELEX, the sequences of 48 clones were sequenced, and sequence convergence was found. Among them, SEQ ID NOs: 17-22 were obtained as representative sequences.

The nucleotide sequences of SEQ ID NOs: 17-22 are shown in the following. Unless particularly indicated, the individual sequences listed in the Examples are shown in the direction of 5′ to 3′, where in each nucleotide, purine (A and G) is 2′-OH (natural RNA type) and pyrimidine (U and C) is a 2′-fluoromodified product. The primer binding sequence is shown in lower case.

SEQ ID NO: 17:
gggagaacuuggagcuGAUGUCUGGAGUCCCCAUAUAUCACGUACAGUG
Uagugcgacgucaguc
SEQ ID NO: 18:
gggagaacuuggagcuCCCCCUCGCACUUAAUGGGUUCUGUGGCUGGAG
AAagugcgacgucaguc
SEQ ID NO: 19:
gggagaacuuggagcuCCCCCUCGCAUUCGGAUUAAUUUGUGACUGCAU
UGagugcgacgucaguc
SEQ ID NO: 20:
gggagaacuuggagcuGGUCCGGAAACUGGAUUCUCUCUAAAAGGGGUA
CCagugcgacgucaguc
SEQ ID NO: 21:
gggagaacuuggagcuCCUGAAUAAGGGCGGGGAAACUUGUGGUGGGCU
AAagugcgacgucaguc
SEQ ID NO: 22:
gggagaacuuggagcuUGACGGCGCUACAUUAUGCUCCAACGGUACUUU
AUagugcgacgucaguc

### [Example 3] Production of RNA Aptamers that Bind Specifically to TGF-β1—3

The same SELEX as in Example 1 was performed using a template having a random sequence of 50 nucleotides and a primer sequence different from that used in Example 1 or 2. TGF-β1 (Recombinant Human TGF-β1, manufactured by Peprotech) immobilized on NHS-activated Sepharose™ 4 Fast Flow (manufactured by GE Healthcare) carrier was used as a target substance of SELEX. The sequences of the template and primers used are shown below. The DNA template and the primers were produced by chemical synthesis.

DNA template sequence:
(SEQ ID NO: 23)
5′-CTGACTCGACGTGCAAGCTTNNNNNNNNNNNNNNNNNNNNNNNNNN
NNNNNNNNNNNNNNNNNNNNNNNNTTGAACACTAGTGCATTCCC-3′
primer Fwd:
(SEQ ID NO: 24)
5′-TAATACGACTCACTATAGGGAATGCACTAGTGTTCAA-3′
primer Rev:
(SEQ ID NO: 25)
5′-CTGACTCGACGTGCAAGCTT-3′

N in the DNA template (SEQ ID NO: 23) is any combination of nucleotides (A, G, C or T). The primer Fwd contains a promoter sequence of T7 RNA polymerase.

After 8 rounds of SELEX, the sequences of 48 clones were sequenced, and sequence convergence was found. Among them, SEQ ID NOs: 26-28 were obtained as representative sequences.

The nucleotide sequences of SEQ ID NOs: 26-28 are shown in the following. Unless particularly indicated, the individual sequences listed in the Examples are shown in the direction of 5′ to 3′, where in each nucleotide, purine (A and G) is 2′-OH (natural RNA type) and pyrimidine (U and C) is a 2′-fluoromodified product. The primer binding sequence is shown in lower case.

SEQ ID NO: 26:
gggaaugcacuaguguucaaCCCCGACCAAUAGCAGCCCGUCUUUAACU
AUUGGAAUCGCAUACGGGCCCaagcuugcacgucgagucag
SEQ ID NO: 27:
gggaaugcacuaguguucaaCAUUUAGCAACACAAGUCGUCCCCCACGG
CAAGCAGUCCUCAAUCCUGACaagcuugcacgucgagucag
SEQ ID NO: 28:
gggaaugcacuaguguucaaUAAACACUAAGUGAUCCUCCUGCAAGCUA
UGAAGAACUUAACGGCUCGUAaagcuugcacgucgagucag

### [Example 4] Measurement of Inhibitory Activity of Aptamer Against TGF-β1-1

Whether the nucleic acids of SEQ ID NOs: 4-13, 17-22, 26-28 inhibit the activity of TGF-β1 was evaluated by a cell assay system that monitors Smad signaling pathway known to be activated by the stimulation of TGF-β. To be specific, photinus luciferase equipped with SBE (Smad-binding element) in the promoter region was used as a reporter (pGL4.48[luc2P/SBE/Hygro] Vector, Promega KK). Together with this SBE-induced photinus luciferase reporter plasmid, a renillaluciferase expression plasmid (pGL4.74[hRluc/TK] Vector, Promega KK) was mixed at a ratio of 20:1 as a standardized control for transfection efficiency, and transfected into HEK293 cells. The transfected HEK293 cells were re-seeded in a 96-well plate and cultured until confluent. TGF-β1 and a mixture of nucleic acid aptamers of SEQ ID NOs: 4-13, 17-22, 26-28 synthesized using T7 RNA polymerase (Y639F) were added thereto to respective final concentrations of 80 pM, 20 nM, and the cells were cultured for 3 hr. Thereafter, the expression levels of photinus luciferase and renillaluciferase were confirmed using Dual-Luciferase (registered trade mark) Reporter Assay System (Promega KK). The measurement value of photinus luciferase in each sample was corrected with the measured value of renillaluciferase, and the relative expression level of each sample was calculated by setting the ratio of photinus luciferase and renillaluciferase in a sample free of addition of TGF-β1 as 1. Furthermore, the relative expression level of a sample added with TGF-β1 alone was defined as inhibition rate 0%, and the relative expression level of a sample free of addition of TGF-β1 was defined as inhibition rate 100%, and the inhibitory activity when each nucleic acid was added was determined. When a nucleic acid pool of 35N, 40N or 50N (SEQ ID NO: 14, 1, 23) was used as a negative control and a known TGF-β antibody (R&D, MAB1835) was used as a positive control, the same treatment and measurement were performed. The results thereof are shown in Table 1.

Measurement of Inhibitory Activity of Aptamer Against TGF-β1-2 Whether or not the nucleic acids of SEQ ID NOs: 6, 9, 19, 21, 26 inhibit the binding of TGF-β1 to the TGF-β receptor was evaluated by surface plasmon resonance method. For the measurement, Biacore T200 manufactured by GE Healthcare was used, and the measurement was performed by the method shown below. About 1500 RU of protein A was immobilized on the surface of a sensor chip of CM4 chip by using an amine coupling kit. 20 μL of Fc fusion TRII receptor (R&D) adjusted to 30 nM was injected as an analyte at a flow rate of 20 μL/min. As a result, the Fc fusion TRII receptor was immobilized on the surface of the sensor chip of the CM4 chip via protein A. Furthermore, a mixed solution of TGF-β1 (4 nM) or TGF-β1 (10 nM), and nucleic acid (30 nM) was injected. Solution A was used as the running buffer. Based on the RU value of a sample injected with TGF-β1 alone and the RU value before injection of TGF-β1, the effect of suppressing an increase in the RU value when TGF-β1 was injected in the presence of nucleic acid was calculated as the effect of inhibiting the binding of TGF-β1 to the TRII receptor. The results in the case of TGF-β1:nucleic acid (Apt)=10 nM: 30 nM are shown in Table 1.

TABLE 1

| | | | cell (luciferase |
|---|---|---|---|
| | bond +++ ≥50 | BIAcore | assay inhibitory |
| | RU ++ 31-50 | inhibition (%) | rate (%)) |
| | RU + 11-30 | TGF-β1:Apt = | TGF-β1:Apt = |
| | RU − ≤10 RU | 10 nM:30 nM | 80 pM:20 nM |
| SEQ ID NO: 1 | − | not tested | 8 |
| SEQ ID NO: 14 | − | not tested | 25 |
| SEQ ID NO: 23 | − | not tested | 0 |
| TGF-β1 | not tested | not tested | 100 (TGF- |
| antibody | | | β1:antibody = |
| | | | 80 pM:2.74 nM) |
| SEQ ID NO: 4 | not tested | not tested | 63 |
| SEQ ID NO: 5 | ++ | not tested | 11 |
| SEQ ID NO: 6 | not tested | 74.98 | 98 |
| SEQ ID NO: 7 | − | not tested | 21 |
| SEQ ID NO: 8 | − | not tested | 22 |
| SEQ ID NO: 9 | not tested | 70.25 | 80 |
| SEQ ID NO: 10 | − | not tested | 15 |
| SEQ ID NO: 11 | ++ | not tested | 32 |
| SEQ ID NO: 12 | − | not tested | 14 |
| SEQ ID NO: 13 | + | not tested | 19 |
| SEQ ID NO: 17 | + | not tested | 7 |
| SEQ ID NO: 18 | ++ | not tested | 16 |
| SEQ ID NO: 19 | not tested | 92.71 | 91 |
| SEQ ID NO: 20 | ++ | not tested | 16 |
| SEQ ID NO: 21 | +++ | 92.59 | 100 |
| SEQ ID NO: 22 | ++ | not tested | 16 |
| SEQ ID NO: 26 | not tested | 84.3 | 81 |
| SEQ ID NO: 27 | + | not tested | −9 |
| SEQ ID NO: 28 | ++ | not tested | −11 |

As shown in Table 1, the nucleic acid consisting of the base sequences shown in SEQ ID NOs: 5, 11, 13, 17, 18, 20, 21, 22, 27 and 28 specifically bound to TGF-β1 (Table 1, left column).

In addition, the nucleic acid consisting of the base sequences shown in SEQ ID NOs: 6, 9, 19, 21 and 26 inhibited the binding of TGF-β1 to TGF-β receptor (Table 1, center column).

Furthermore, the nucleic acid consisting of the base sequences shown in SEQ ID NOs: 4, 6, 9, 19, 21 and 26 showed strong inhibitory activity in the cell assay system (Table 1, right column). On the other hand, the nucleic acid consisting of the base sequences shown in SEQ ID NOs: 5, 7-8, 10-13, 17-18, 20, 22, 27-28 did not show inhibitory activity against TGF-β1 in the cell assay system.

What should be noted in this result is that SEQ ID NOs: 4, 6, 9, and 21 contained a common sequence. The sequence is referred to as common sequence 1 in the present specification.

common sequence 1:
UAAXGGRBGGSGARACUUGKGVBRGG

(X is a bond or GU; R is A or G; S is C or G; K is G or U; V is A, C, or G; and B is C, G, or U)

An aptamer having the nucleotide sequence represented by the above-mentioned common sequence 1 shows remarkably strong inhibitory activity against TGF-β1, as shown in Table 1.

In other words, an aptamer having the nucleotide sequence represented by common sequence 1 was considered to be an aptamer that specifically and remarkably strongly binds to TGF-β1. In addition, an aptamer having the nucleotide sequence represented by common sequence 1 was considered to be highly likely an aptamer capable of inhibiting the activity of TGF-β1.

### [Example 5] Shortening of Aptamer

The aptamer of SEQ ID NO: 21 was shortened. The sequences of these short chained forms are shown in SEQ ID NOs: 29-31.

Respective nucleotide sequences are shown below. The common sequence 1 found in Examples 1-4 is underlined. Unless particularly indicated, the individual sequences listed in the Examples are shown in the direction of 5′ to 3′, and in each nucleotide, purine (A and G) is 2′-OH (natural RNA type) and pyrimidine (U and C) is a 2′-fluoromodified product. The primer binding sequence is shown in lower case.

SEQ ID NO: 29: (sequence obtained by shortening
aptamer shown in SEQ ID NO: 21 to a length of 51
nucleotides containing the common sequence)
gggagaacuuggagcuCCUGAAUAAGGGCGGGGAAACUUGUGGUGGGCU
AA
SEQ ID NO: 30: (sequence obtained by shortening
aptamer shown in SEQ ID NO: 21 to a length of 26
nucleotides containing a part of the common
sequence)
GGGCGGGGAAACUUGUGGUGGGCUAA
SEQ ID NO: 31: (sequence obtained by shortening
aptamer shown in SEQ ID NO: 21 to a length of 33
nucleotides containing the common sequence)
ggcAUAAGGGCGGGGAAACUUGUGGUGGGCUAA

The nucleic acids of SEQ ID NOs: 29-31 were obtained by transcription using T7 RNA polymerase (Y639F) and using a chemically-synthesized DNA sequence containing a promoter sequence of T7 RNA polymerase as a template.

Whether or not these nucleic acids inhibit the binding of TGF-β1 to the TGF-β receptor was determined by injecting a mixed solution of TGF-β1 (4 nM) or TGF-β1 (10 nM) and nucleic acid (30 nM) in the same manner as in Example 4, and evaluating by the surface plasmon resonance method. The measurement results in the case of TGF-β1:nucleic acid (Apt)=10 nM:30 nM (1:3) are shown in Table 2.

The nucleic acids of SEQ ID NOs: 21, 29, 31 were obtained by transcription using T7 RNA polymerase (Y639F) and using a chemically-synthesized DNA sequence containing a promoter sequence of T7 RNA polymerase as a template. Whether or not these nucleic acids inhibit the activity of TGF-β1 was evaluated by a luciferase reporter assay as in Example 4. The measurement results are shown in Table 2. In Table 2, the results of two independent tests are also shown for the value of the luciferase assay inhibition rate.

TABLE 2

| | | | cell (luciferase |
|---|---|---|---|
| | bond +++ ≥50 | BIAcore | assay inhibitory |
| | RU ++ 31-50 | inhibition (%) | rate (%)) |
| | RU + 11-30 | TGF-β1:Apt = | TGF-β1:Apt = |
| | RU − ≤10 RU | 1:3 | 80 pM:20 nM |
| SEQ ID NO: 21 | +++ | 74.54 | 96/95 |
| SEQ ID NO: 29 | not tested | 75.86 | 91/94 |
| SEQ ID NO: 30 | − | −5.84 | not tested |
| SEQ ID NO: 31 | not tested | 56.23 | 79/87 |

As shown in Table 2, the inhibitory activity against TGF-β1 was maintained even when the sequences near the common sequence were removed (SEQ ID NOs: 29, 31). On the other hand, the inhibitory activity against TGF-β1 was markedly lowered when even the common sequence was removed (SEQ ID NO: 30). From the above, it was shown that common sequence 1 is important for exhibiting the binding activity to TGF-β1 and the inhibitory activity against TGF-β1.

### [Example 6] Production of Highly Active TGF-β1 Aptamer—1

SELEX was performed using an RNA pool in which a part of the common sequence 1 was made into a random sequence in the sequence shown in SEQ ID NO: 31. SELEX was performed in the same manner as in Example 1. The template and the primer sequence on the 5′-terminal side are shown below. In addition, the nucleic acid of SEQ ID NO: 16 was used as the primer Rev.

DNA template sequence:
(SEQ ID NO: 32)
5′-gggagaacttggagctcctgaNNNNGGGNNGGGNNNNNNGTGGNGG
GNNNNagtgcgacgtcagtc-3′
primer Fwd:
(SEQ ID NO: 33)
5′-TAATACGACTCACTATAGGGAGAACTTGGAGCTCCTGA-3′

Each DNA library pool after the completion of rounds 0 to 6 was examined with a high-throughput sequencer (IonPGM™ system, Thermo Fisher Scientific). As a result, the sequences after 2R had common sequence 2 (SEQ ID NO: 34). Among them, a sequence having the largest number of reads (SEQ ID NO: 35), a sequence having the next largest number (SEQ ID NO: 36), sequences different in one base (SEQ ID NO: 37, 38) from the sequence having the largest number of reads (SEQ ID NO: 35), a sequence in which one base of common sequence 2 in SEQ ID NO: was deleted (SEQ ID NO: 39), a sequence in which one base was deleted at a position (last GGG on 3′-terminal side) different from that in SEQ ID NO: 39 (SEQ ID NO: 41), and a sequence when the deletion did not occur (SEQ ID NO: 40) are shown below. SEQ ID NOs: 35-40 were all detected in this experiment (Example 6). Unless particularly indicated, the individual sequences listed in the Examples are shown in the direction of 5′ to 3′, where in each nucleotide, purine (A and G) is 2′-OH (natural RNA type) and pyrimidine (U and C) is a 2′-fluoromodified product. The primer binding sequence is shown in lower case.

SEQ ID NO: 34: (common sequence 2 obtained in
this experiment)
AUAAGGGHGGGGAGACUUGUGGWGGG
(H is A, C, or U; and W is A or U)

Respective nucleotide sequences are shown below. The part corresponding to common sequence 2 (SEQ ID NO: 34) is underlined. Unless particularly indicated, the individual sequences listed in the Examples are shown in the direction of 5′ to 3′, where in each nucleotide, purine (A and G) is 2′-OH (natural RNA type) and pyrimidine (U and C) is a 2′-fluoromodified product. The primer binding sequence is shown in lower case.

SEQ ID NO: 35: (sequence having the largest number
of reads detected with high-throughput sequencer)
gggagaacuuggagcuccugaAUAAGGGAGGGGAGACUUGUGGAGGGCA
AGagugcgacgucaguc
SEQ ID NO: 36: (sequence having the secon largest
number of reads detected with high-throughput
sequencer)
gggagaacuuggagcuccugaAUAAGGGAGGGGAGACUUGUGGAGGGCA
AAagugcgacgucaguc
SEQ ID NO: 37: (sequence different in one base
from the sequence having the largest number of
reads (SEQ ID NO: 35))
gggagaacuuggagcuccugaAUAAGGGAGGGGAGACUUGUGGUGGGCA
AGagugcgacgucaguc
SEQ ID NO: 38: (sequence different in one base
from the sequence having the largest number of
reads (SEQ ID NO: 35))
gggagaacuuggagcuccugaAUAAGGGUGGGGAGACUUGUGGAGGGCA
AGagugcgacgucaguc
SEQ ID NO: 39: (sequence in which one base of
common sequence 2 in SEQ ID NO: 35 was deleted)
gggagaacuuggagcuccugaAUAAGGGAGGGAGACUUGUGGAGGGCAA
Gagugcgacgucaguc
SEQ ID NO: 40: (sequence when the deletion in
SEQ ID NO: 41 did not occur)
gggagaacuuggagcuccugaAUAAGGGAGGGGAGACUUGUGGAGGGCA
GAagugcgacgucaguc
SEQ ID NO: 41: (sequence in which one base was
deleted from

last GGG on 3′-terminal side in common sequence 2 in SEQ ID NO: 35)

gggagaacuuggagcuccugaAUAAGGGAGGGGAGACUUGUGGAGGCAGA
agugcgacgucaguc

The nucleic acids of SEQ ID NOs: 35-41 were obtained by transcription using T7 RNA polymerase (Y639F) and using a chemically-synthesized DNA sequence containing a promoter sequence of T7 RNA polymerase as a template. Whether or not these nucleic acids inhibit the binding of TGF-β1 to the TGF-β receptor was determined by injecting a mixed solution of TGF-β1 (10 nM) and nucleic acid (10 nM or 30 nM) in the same manner as in Example 4, and evaluating by the surface plasmon resonance method. The measurement results in the case of TGF-β1:nucleic acid (Apt)=10 nM:30 nM, 10 nM:10 nM are shown in Table 3.

Whether or not these nucleic acids inhibit the activity of TGF-β1 was evaluated by a luciferase reporter assay similar to that in Example 4, as in Example 1. The measurement results are shown in Table 3.

TABLE 3

| | | | | cell (luciferase |
|---|---|---|---|---|
| | bond +++ ≥50 | BIAcore | BIAcore | assay inhibitory |
| | RU ++ 31-50 | inhibition (%) | inhibition (%) | rate (%)) |
| | RU + 11-30 | TGF-β1:Apt = | TGF-β1:Apt = | TGF-β1:Apt = |
| | RU − ≤10 RU | 10 nM:30 nM | 10 nM:10 nM | 80 pM:20 nM |
| SEQ ID NO: 35 | +++ | 98.8 | 51.3 | 100 |
| SEQ ID NO: 36 | not tested | 100.5 | 45.5 | 101 |
| SEQ ID NO: 37 | not tested | 97.8 | 45.8 | 100 |
| SEQ ID NO: 38 | not tested | 95.4 | 43.4 | 100 |
| SEQ ID NO: 39 | − | 5 | 1.9 | 15 |
| SEQ ID NO: 40 | +++ | 77.6 | 38 | 100 |
| SEQ ID NO: 41 | − | 35.4 | 12.3 | 39 |

As shown in Table 3, aptamers containing the nucleotide sequence represented by the common sequence 2 (SEQ ID NO: 34) showed inhibitory activity against TGF-β1 (SEQ ID NOs: 35-38, 40). On the other hand, an aptamer in which a part of common sequence 2 (SEQ ID NO: 34) was deleted did not show inhibitory activity against TGF-β1 (SEQ ID NOs: 39, 41). From these results, it was clarified that an aptamer having a nucleotide sequence shown in common sequence 2 (SEQ ID NO: 34) binds to TGF-β1 and inhibits TGF-β1 activity.

### [Example 7] Shortening and Base substitution of highly active TGF-β1 Aptamer

The sequence shown in SEQ ID NO: 35 was shortened by reference to SEQ ID NO: 31 to obtain SEQ ID NO: 42. In addition, in order to determine the optimum bases at two positions considered to be highly variable from the results of the high-throughput sequence, sequences based on SEQ ID NO: 42 and substituted with other bases were prepared (SEQ ID NOs: 43-47). By reference to the results of the high-throughput sequence, among the sequences having a relatively large number of detected reads, SEQ ID NO: 48 having a pattern different from that of SEQ ID NOs: 43-47 was produced.

The respective nucleotide sequences are shown below. Unless particularly indicated, the individual sequences listed in the Examples are shown in the direction of 5′ to 3′, where in each nucleotide, purine (A and G) is 2′-OH (natural RNA type) and pyrimidine (U and C) is a 2′-fluoromodified product. [ ] indicates nucleotides considered to be highly variable.

SEQ ID NO: 42: (sequence obtained by shortening
aptamer shown in SEQ ID NO: 35 to a length of 33
nucleotides by reference to SEQ ID NO: 32)
GGCAUAAGGG[A]GGGGAGACUUGUGG[A]GGGCAAG
SEQ ID NO: 43: (sequence in which the 11th A of
the aptamer shown in SEQ ID NO: 42 was replaced
with U)
GGCAUAAGGG[U]GGGGAGACUUGUGG[A]GGGCAAG
SEQ ID NO: 44: (sequence in which the 11th A of
the aptamer shown in SEQ ID NO: 42 was replaced
with C)
GGCAUAAGGG[C]GGGGAGACUUGUGG[A]GGGCAAG
SEQ ID NO: 45: (sequence in which the 26th A of
the aptamer shown in SEQ ID NO: 42 was replaced
with U)
GGCAUAAGGG[A]GGGGAGACUUGUGG[U]GGGCAAG
SEQ ID NO: 46: (sequence in which the 11th A was
replaced with U and the 26th A was replaced with
U in the aptamer shown in SEQ ID NO: 42)
GGCAUAAGGG[U]GGGGAGACUUGUGG[U]GGGCAAG
SEQ ID NO: 47: (sequence in which the 11th A was
replaced with C and the 26th A was replaced with
U in the aptamer shown in SEQ ID NO: 42)
GGCAUAAGGG[C]GGGGAGACUUGUGG[U]GGGCAAG
SEQ ID NO: 48: (sequence similar to SEQ ID NO: 42
and obtained by designing a sequence in which the
26th A was C by reference to the results of high-
throughput sequence)
GGCAUAAGGG[A]GGGGAGACUUGUGG[C]GGGUAAA

The nucleic acids of SEQ ID NOs: 42-48 used were chemically synthesized and purified by HPLC. Whether or not these nucleic acids inhibit the binding of TGF-β1 to the TGF-β receptor was determined by injecting a mixed solution of TGF-β1 (4 nM) and nucleic acid (4 nM) in the same manner as in Example 4, and evaluating by the surface plasmon resonance method. The measurement results in the case of TGF-β1:nucleic acid (Apt)=4 nM:4 nM (1:1) are shown in Table 4.

Whether or not these nucleic acids inhibit the activity of TGF-β1 was evaluated by adding to the final concentrations of TGF-β1 (80 pM) and nucleic acid (312.5 pM), and by a luciferase reporter assay as in Example 4. The measurement results are shown in Table 4.

TABLE 4

| | | cell (luciferase |
|---|---|---|
| | BIAcore | assay inhibitory |
| | inhibition (%) | rate (%)) |
| | TGF-β1:Apt = | TGF-β1:Apt = |
| | 1:1 | 80 pM:312.5 pM |
| SEQ ID NO: 42 | 98.97 | 82.4 |
| SEQ ID NO: 43 | 98.97 | 71.5 |
| SEQ ID NO: 44 | 98.71 | 69.4 |
| SEQ ID NO: 45 | 99.23 | 65.5 |
| SEQ ID NO: 46 | 99.23 | 57.3 |
| SEQ ID NO: 47 | 99.23 | 70.3 |
| SEQ ID NO: 48 | 93.55 | 63.9 |

As shown in Table 4, aptamers containing the nucleotide sequence represented by the common sequence 2 (SEQ ID NO: 34) showed inhibitory activity against TGF-β1 (SEQ ID NO: 42) even when the chain was shortened.

From the results of the high-throughput sequence, even when two bases considered to be highly variable (base [A] (H in common sequence 2) between the first G set and the second G set, and the base [A] (W in common sequence 2)) between the third G set and the fourth G set were altered, the inhibitory activity against TGF-β1 was maintained (SEQ ID NO: 42-47). Also, the inhibitory activity against TGF-β1 was maintained even when CAGG after the fourth G set was replaced with other sequence (SEQ ID NO: 48). From the above, it was shown that common sequence 2 (SEQ ID NO: 34) is important for exhibiting the binding activity to TGF-β1 and the inhibitory activity against TGF-β1.

### [Example 8] Optimization of Chemical Modification of Highly Active Shortened Aptamer—1

Based on the sequence shown in SEQ ID NO: 42, the aptamer was modified to a chemically-modified product in which the nucleotide ribose was 2′-OMe, and the effect of the aptamer on the TGF-β1 inhibitory activity was investigated. The prepared variants are shown in SEQ ID NOs: 42(1)-42(3). In each nucleotide, purine (A and G) is 2′-OH (natural RNA type) and the base denoted as (F) is a 2′-fluoromodified product. The base denoted as (M) is a 2′-OMe-modified product. The individual sequences listed in the Examples are shown in the direction of 5′ to 3′.

SEQ ID NO: 42(1): (sequence in which the first to
the third bases of the aptamer shown in SEQ ID
NO: 42 were replaced with OMe-modified bases)
G(M)G(M)C(M)AU(F)AAGGGAGGGGAGAC(F)U(F)U(F)GU(F)
GGAGGGC(F)AAG
SEQ ID NO: 42(2): (sequence obtained by replacing
the 11th base of the aptamer shown in SEQ ID
NO: 42 with OMe-modified base)
GGC(F)AU(F)AAGGGA(M)GGGGAGAC(F)U(F)U(F)GU(F)GGAGG
GC(F)AAG
SEQ ID NO: 42(3): (sequence obtained by replacing
the 26th base of the aptamer shown in SEQ ID
NO: 42 with OMe-modified base)
GGC(F)AU(F)AAGGGAGGGGAGAC(F)U(F)U(F)GU(F)GGA(M)GGG
C(F)AAG

The nucleic acids of SEQ ID NOs: 42(1)-42(3) used were chemically synthesized and purified by HPLC. Whether or not these nucleic acids inhibit the binding of TGF-β1 to the TGF-β receptor was determined by injecting a mixed solution of TGF-β1 (4 nM) and nucleic acid (4 nM) in the same manner as in Example 4, and evaluating by the surface plasmon resonance method. The measurement results in the case of TGF-β1:nucleic acid (Apt)=4 nM:4 nM (1:1) are shown in Table 5.

Whether or not these nucleic acids inhibit the activity of TGF-β1 was evaluated by adding to the final concentrations of TGF-β1 (80 pM) and nucleic acid (312.5 pM), and by a luciferase reporter assay as in Example 4. The measurement results are shown in Table 5.

TABLE 5

| | | cell (luciferase |
|---|---|---|
| | BIAcore | assay inhibitory |
| | inhibition (%) | rate (%)) |
| | TGF-β1:Apt = | TGF-β1:Apt = |
| | 1:1 | 80 pM:312.5 pM |
| SEQ ID NO: 42 | 99.69 | 68% |
| SEQ ID NO: 42(1) | 95.67 | 22% |
| SEQ ID NO: 42(2) | 98.45 | 88% |
| SEQ ID NO: 42(3) | 98.76 | 87% |

As shown in Table 5, in the aptamer of the present invention, it was found that OME modification is possible for sequences (SEQ ID NO: 42 (1)) other than the nucleotide sequence represented by common sequence 2 (SEQ ID NO: 34).

On the other hand, it was found that, in the nucleotide sequence shown in common sequence 2 (SEQ ID NO: 34), the two bases considered to be highly variable from the results of the high-throughput sequence, as examined in Example 7 (Table 4), permit OMe modification (SEQ ID NOs: 42(2), 42(3)).

### Optimization of Chemical Modification of Highly Active Shortened Aptamer—2

Based on the sequence shown in SEQ ID NO: 42(2), the aptamer was substituted with a modified base in which the nucleotide ribose was 2′-OMe, and the effect of the aptamer on the TGF-β1 inhibitory activity was investigated. The sequences of the mutant aptamers thus produced are shown in SEQ ID NOs: 42(2-1)-42(2-8). The substituted nucleotides are underlined. In each nucleotide, purine (A and G) is 2′-OH (natural RNA type) and the base denoted as (F) is a 2′-fluoromodified product. The base denoted as (M) is a 2′-OMe-modified product. The individual sequences listed in the Examples are shown in the direction of 5′ to 3′.

SEQ ID NO: 42(2-1): (sequence obtained by
replacing the 26th base of the aptamer shown in
SEQ ID NO: 42(2) with OMe-modified base)
GGC(F)AU(F)AAGGGA(M)GGGGAGAC(F)U(F)U(F)GU(F)GGA
(M)GGGC(F)AAG
SEQ ID NO: 42(2-2): (sequence obtained by
replacing the first base of the aptamer shown in
SEQ ID NO: 42(2) with OMe-modified base)
G(M)GC(F)AU(F)AAGGGA(M)GGGGAGAC(F)U(F)U(F)GU(F)GG
AGGGC(F)AAG
SEQ ID NO: 42(2-3): (sequence obtained by
replacing the 4th base of the aptamer shown in
SEQ ID NO: 42(2) with OMe-modified base)
GGC(F)A(M)U(F)AAGGGA(M)GGGGAGAC(F)U(F)U(F)GU(F)GG
AGGGC(F)AAG
SEQ ID NO: 42(2-4): (sequence obtained by
replacing the 16th base of the aptamer shown in
SEQ ID NO: 42(2) with OMe-modified base)
GGC(F)AU(F)AAGGGA(M)GGGGA(M)GAC(F)U(F)U(F)GU(F)GG
AGGGC(F)AAG
SEQ ID NO: 42(2-5): (sequence obtained by
replacing the 17th base of the aptamer shown in
SEQ ID NO: 42(2) with OMe-modified base)
GGC(F)AU(F)AAGGGA(M)GGGGAG(M)AC(F)U(F)U(F)GU(F)GG
AGGGC(F)AAG
SEQ ID NO: 42(2-6): (sequence obtained by
replacing the 31st base of the aptamer shown in
SEQ ID NO: 42(2) with OMe-modified base)
GGC(F)AU(F)AAGGGA(M)GGGGAGAC(F)U(F)U(F)GU(F)GGAGG
GC(F)A(M)AG
SEQ ID NO: 42(2-7): (sequence obtained by
replacing the 32nd base of the aptamer shown in
SEQ ID NO: 42(2) with OMe-modified base)
GGC(F)AU(F)AAGGGA(M)GGGGAGAC(F)U(F)U(F)GU(F)GGAGG
GC(F)AA(M)G
SEQ ID NO: 42(2-8): (sequence obtained by
replacing the 33rd base of the aptamer shown in
SEQ ID NO: 42(2) with OMe-modified base)
GGC(F)AU(F)AAGGGA(M)GGGGAGAC(F)U(F)U(F)GU(F)GGAGG
GC(F)AAG(M)

The nucleic acids of SEQ ID NOs: 42(2-1)-42(2-8) used were chemically synthesized and purified by HPLC. Whether or not these nucleic acids inhibit the binding of TGF-β1 to the TGF-β receptor was determined by injecting a mixed solution of TGF-β1 (4 nM) and nucleic acid (4 nM) in the same manner as in Example 4, and evaluating by the surface plasmon resonance method. The measurement results in the case of TGF-β1:nucleic acid (Apt)=4 nM:4 nM (1:1) are shown in Table 6. Whether or not these nucleic acids inhibit the activity of TGF-β1 was evaluated by adding to the final concentrations of TGF-β1 (80 pM) and nucleic acid (312.5 pM), and by a luciferase reporter assay as in Example 4. The measurement results are shown in Table 6.

TABLE 6

| | | cell (luciferase assay |
|---|---|---|
| | BIAcore | inhibitory rate (%)) |
| | inhibition (%) | TGF-β1:Apt = 80 |
| | TGF-β:Apt = 1:1 | pM:312.5 pM |
| SEQ ID NO: 42 | 81.66 | 44% |
| SEQ ID NO: 42(2) | 71.32 | 73% |
| SEQ ID NO: 42(3) | 80.88 | 64% |
| SEQ ID NO: 42(2-1) | 75.22 | 61% |
| SEQ ID NO: 42(2-2) | 76 | 66% |
| SEQ ID NO: 42(2-3) | 70.73 | 66% |
| SEQ ID NO: 42(2-4) | 84.2 | 64% |
| SEQ ID NO: 42(2-5) | 75.61 | 62% |
| SEQ ID NO: 42(2-6) | 81.85 | 72% |
| SEQ ID NO: 42(2-7) | 78.93 | 78% |
| SEQ ID NO: 42(2-8) | 83.02 | 72% |

From the results of Table 6, it was found that, in the nucleotide sequence shown in common sequence 2 (SEQ ID NO: 34), (1) the two bases considered to be highly variable from the results of the high-throughput sequence, as examined in Example 7 (Table 4), both permit OMe modification (SEQ ID NO: 42(2-1)) (2) the respective bases AUAA and AGACUU also permit OMe modification (SEQ ID NO: 42(2-4) and 42(2-5)).

### INDUSTRIAL APPLICABILITY

The aptamer of the present invention can be useful as a medicament for preventing and/or treating various diseases involving activation of TGF-β1 such as fibrosis, cancer, and the like, or a diagnostic reagent or a labeling agent.

This application is based on a patent application No. 2019-126940 filed in Japan (filing date: Jul. 8, 2019), the contents of which are incorporated in full herein.

1. An aptamer that binds to TGF-β1, comprising four sets of consecutive G bases, and a combination of nucleotide sequences represented by the following formula (I) and the formula (II):
UAAX formula (I):
ARACUU formula (II):
wherein X is a bond or GU; and R is A or G.

2. The aptamer according to claim 1, the nucleotide sequence represented by the formula (I): UAAX is located on the most N terminal side of the four sets of G bases, and the nucleotide sequence represented by the formula (II): ARACUU is located between the second set of G bases and the third set of G bases.

3. The aptamer according to claim 1, comprising a nucleotide sequence represented by the following formula (III):
formula (III):
UAAXGGRNGGSGARACUUGKGVNRGG
wherein X is a bond or GU; N is any base; R is A or G; S is C or G; K is G or U; V is A, C, or G; and B is C, G, or U (only in combination that forms four sets of G bases).

4. The aptamer according to claim 1, comprising a nucleotide sequence represented by the following formula (III′):
formula (III′):
UAAXGGRBGGSGARACUUGKGVBRGG
wherein X is a bond or GU; R is A or G; S is C or G; K is G or U; V is A, C, or G; and B is C, G, or U (only in combination that forms four sets of G bases).

5. The aptamer according to claim 1, comprising a nucleotide sequence represented by the following formula (III″):
formula (III″):
AUAAGGGHGGGGAGACUUGUGGWGGG
wherein W is A or U; and H is A, C, or U.

6. The aptamer according to claim 1, wherein at least one nucleotide contained in the aptamer is modified.

7. An aptamer that binds to TGF-β1, comprising the nucleotide sequence of any of the following (a)-(c):
(a) the sequence shown in SEQ ID NO: 4-6, 9, 11, 13, 17-22, 26-29 or 31;
(b) the sequence of the above-mentioned (a), wherein one to several nucleotides are substituted, deleted, inserted, or added; or,
(c) the sequence of the above-mentioned (a) or (b), wherein at least one nucleotide is modified.

8. The aptamer according to claim 1, having a nucleotide length of not more than 55.

9. The aptamer according to claim 1, that inhibits binding between TGF-β1 and a TGF-β1 receptor.

10. A complex comprising the aptamer according to claim 1 and a functional substance.

11. A medicament comprising the aptamer according to claim 1.

12. A method for detecting TGF-β1, comprising using the aptamer according to claim 1.

13. The aptamer according to claim 7, that inhibits binding between TGF-β1 and a TGF-β1 receptor.
